(19) United States
(12) Patent Application Publication
(10) Pub. No.: US 2024/0417784 A1
(43) Pub. Date: Dec. 19, 2024

(54) METHODS, COMPOSITIONS, KITS, AND SYSTEMS FOR ENHANCING ANALYTE CAPTURE FOR SPATIAL ANALYSIS

(71) Applicant: 10x Genomics, Inc., Pleasanton, CA (US)

(72) Inventors: David Sukovich, Oakland, CA (US); Hanyoup Kim, Foster City, CA (US); Jerald Sapida, San Leandro, CA (US); Spontaneous Rose McKnight Russell, San Ramon, CA (US); Layla Katiraee, Castro Valley, CA (US); Augusto Manuel Tentori, Dublin, CA (US)

(21) Appl. No.: 18/815,241

(22) Filed: Aug. 26, 2024

Related U.S. Application Data
(62) Division of application No. 18/311,605, filed on May 3, 2023, now Pat. No. 12,071,655.
Application No. 18/311,605 is a continuation of application No. PCT/US2022/031923, filed on Jun. 2, 2022.
(60) Provisional application No. 63/196,435, filed on Jun. 3, 2021.

(51) Int. Cl. C12Q 1/6841; C12N 15/10

(52) U.S. Cl. CPC C12Q 1/6841; C12N 15/1065

(57) ABSTRACT

Provided herein are methods of enhancing spatial resolution of an analyte using sandwich maker system. The methods and systems used herein include a first substrate that includes a plurality of probes that include a capture domain and a spatial domain and a second substrate that includes a plurality of probes comprising a capture domain and a spatial domain.

### CROSS-REFERENCE TO RELATED APPLICATIONS

This application is a divisional application of application Ser. No. 18/311,605, filed on May 3, 2023, which is a continuation of International Application PCT/US2022/031923, with an international filing date of Jun. 2, 2022, which claims the benefit of U.S. Provisional Application Ser. No. 63/196,435, filed Jun. 3, 2021, the entire contents of which are incorporated by reference herein.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Preserving the original spatial distribution of gene expression (also referred to as resolution) is critical for spatial transcriptomics gene expression assays. One option to analyze gene expression is by using multiple substrates. For example, one substrate (e.g., slide) could comprise a barcoded array while another includes a biological specimen (such as a tissue section). This set up provides the ability to capture analytes on one substrate. And while capturing analytes on one substrate provides spatial information, there remains a need to increase resolution and/or sensitivity in spatial methods that incorporate a system that utilizes capture probes on multiple substrates.

### SUMMARY

Disclosed herein are methods and systems utilizing multiple substrates (e.g., slides) to assess spatial heterogeneity of analytes in a sample. The methods and systems disclosed herein increase the resolution and/or sensitivity of analyte detection. The methods and systems here utilize multiple substrate arrays, which include at least two slides on either side of a biological sample. Featured herein are methods and systems that include a first slide that includes a plurality of probes comprising a first capture probe and a first spatial barcode. The methods here also feature a second slide which also includes an array of capture probes that include at least a capture domain sequence and spatial barcode. In some instances, the biological sample is provided on the first slide; and after permeabilizing the biological sample, analytes are free to disperse from the biological sample. Because the analytes passively diffuse, they will be captured by the probes on both the first and second slides, thus increasing the total number of analytes captured at a particular spot (e.g., or i.e., compared to a system that utilizes one substrate).

In one aspect of the disclosure, provided herein is a method for determining the abundance and location of multiple analytes in a biological sample. In some instances, the method comprises: (a) providing a first substrate and a biological sample mounted thereon, wherein the first substrate comprises a plurality of first capture probes, wherein a first capture probe of the plurality of first capture probes comprises (i) a first spatial barcode and (ii) a first capture domain; (b) aligning a second substrate on the opposite side of the first substrate relative to the biological sample, thereby sandwiching the first substrate, the biological sample, and the second substrate, wherein the second substrate comprises a plurality of second capture probes, wherein a second capture probe of the plurality of second capture probes comprises (i) a second spatial barcode and (ii) a second capture domain; and (c) hybridizing a first analyte of the multiple analytes to the first capture domain and hybridizing a second analyte of the multiple analytes to the second capture domain. In some instances, the method further comprises (d) (A) determining (i) all or a portion of the sequence of the first spatial barcode, or a complement thereof, and (ii) all or a portion of the sequence of the first analyte captured on the first capture domain, or a complement thereof, and using the sequences of (i) and (ii) to determine the abundance and the location of the first analyte in the biological sample, and/or (B) determining (i) all or a portion of the sequence of the second spatial barcode, or a complement thereof, and (ii) all or a portion of the sequence of the second analyte captured on the second capture domain, or a complement thereof, and using the sequences of (i) and (ii) to determine the abundance and the location of the second analyte in the biological sample.

In some instances, the methods further comprise, prior to (a) mounting the biological sample onto the plurality of first capture probes of the first substrate.

In some instances, total number of the multiple analytes determined is increased by about 10%, about 20%, about 30%, about 40%, about 50%, about 60%, about 70%, about 80%, about 90%, about 100%, or more compared to a method using one substrate.

In some instances, the methods further include adding a permeabilization buffer to the biological sample, thereby promoting migration of the first analyte and the second analyte from the biological sample. In some instances, the permeabilization buffer comprises pepsin or proteinase K.

In some instances, step (b) of the above methods is performed with the aid of a sample holder comprising: (i) a first member comprising a first retaining mechanism configured to receive the first substrate, (ii) a second member configured to receive the second substrate, and (iii) an alignment mechanism that is connected to at least one of the first member and second member and configured to align the first substrate and the second substrate. In some instances, step (b) of the above methods comprises (i) retaining the first substrate in the first retaining mechanism of the first substrate, (ii) retaining the second substrate in the second retaining mechanism of the second substrate, and (iii) using the alignment mechanism to align the second substrate on the opposite side of the first substrate relative to the biological sample, thereby sandwiching the first substrate, the biological sample, and the second substrate.

In some instances, the first capture domain comprises (i) a poly-thymine (poly(T)) sequence, (ii) a sequence complementary to a capture handle sequence present in an analyte capture agent, or (iii) a sequence complementary to a portion of a connected probe generated by templated ligation. In some instances, the plurality of first capture probes is arranged on a plurality of first beads. In some instances, the first capture probe further comprises one or more first functional domains, a first unique molecular identifier, a first cleavage domain, and combinations thereof.

In some instances, the methods further include generating a first extended capture probe using the first analyte as a template. In some instances, the first capture probe is extended at its 3′ end. In some instances, the generating the first extended capture probe utilizes a reverse transcriptase. In some instances, the generating the first extended capture probe utilizes fluorescently labeled nucleotides.

In some instances, the methods further include amplifying the extended capture probe to produce a plurality of extended capture probes. In some instances, the amplifying utilizes a DNA polymerase, a plurality of primers, and a plurality of nucleotides.

In some instances, the determining step in step (d) (A) in the above methods comprises sequencing. In some instances, the determining step in step (d) (A) comprises sequencing (i) all or a portion of the sequence of the first spatial barcode or the complement thereof, and (ii) all or a portion of the sequence of the first analyte.

In some instances, the second capture domain comprises (i) a poly-thymine (poly(T)) sequence, (ii) a sequence complementary to a capture handle sequence present in an analyte capture agent, or (iii) a sequence complementary to a portion of a connected probe generated by templated ligation. In some instances, the plurality of second capture probes is arranged on a plurality of second beads. In some instances, the second capture probe further comprises one or more second functional domains, a second unique molecular identifier, a second cleavage domain, and combinations thereof.

In some instances, the methods disclosed herein further include generating a second extended capture probe using the second analyte as a template. In some instances, the second capture probe is extended at its 3′ end. In some instances, the generating the second extended capture probe utilizes a reverse transcriptase. In some instances, the generating the second extended capture probe utilizes fluorescently labeled nucleotides.

In some instances, the methods above further include amplifying the extended capture probe to produce a plurality of extended capture probes. In some instances, the amplifying utilizes a DNA polymerase, a plurality of primers, and a plurality of nucleotides.

In some instances, the determining step in step (d) (B) in the methods above comprises sequencing. In some instances, the determining step in step (d) (B) comprises sequencing (i) all or a portion of the sequence of the second spatial barcode or the complement thereof, and (ii) all or a portion of the sequence of the second analyte.

In some instances, the first analyte and the second analyte are RNA molecules. In some instances, the RNA molecules are mRNA molecules. In some instances, the first analyte and the second analyte are different analytes. For example, the first analyte and the second analyte can be a protein and a nucleic acid (e.g., RNA or DNA). Additionally, or in the alternative, the first analyte and the second analyte can be an intermediate agent or portion thereof and RNA. The intermediate agent can be a connected probe (e.g., RTL probe) or analyte capture agent (e.g., oligo-conjugated antibody). Additionally, or in the alternative, the first analyte and the second analyte can be RNA and DNA. Additionally, or in the alternative, the first analyte and the second analyte can be an intermediate agent and DNA. Additionally, or in the alternative, the first analyte and the second analyte can be a targeted capture on one slide and whole transcriptome or DNA on other slide.

In some instances, the biological sample is a tissue section sample. In some instances, the biological sample is a tissue section. In some instances, the biological sample is a formalin-fixed, paraffin-embedded (FFPE) sample, a frozen sample, a fresh frozen sample, or a fresh sample. In some instances, the biological sample is an FFPE sample.

Also disclosed herein are kits. In some instances, the kits include (a) a first substrate comprising a plurality of first capture probes, wherein a first capture probe of the plurality of first capture probes comprises (i) a first spatial barcode and (ii) a first capture domain; (b) a second substrate comprises a plurality of second capture probes, wherein a second capture probe of the plurality of second capture probes comprises (i) a second spatial barcode and (ii) a second capture domain; and (c) instructions for performing any of the above methods.

Also disclosed herein are systems. In some instances, the systems are used for determining the abundance and location of multiple analytes in a biological sample. In some instances, the systems include (a) a first substrate comprising a plurality of first capture probes, wherein a first capture probe of the plurality of first capture probes comprises (i) a first spatial barcode and (ii) a first capture domain; (b) a second substrate comprises a plurality of second capture probes, wherein a second capture probe of the plurality of second capture probes comprises (i) a second spatial barcode and (ii) a second capture domain; (c) a biological sample holder comprising: (i) a first member comprising a first retaining mechanism configured to receive the first substrate, (ii) a second member configured to receive the second substrate, and (iii) an alignment mechanism that is connected to at least one of the first member and second member and configured to align the first substrate and the second substrate; and (d) the biological sample.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce spatially-barcoded cells or cellular contents.

FIG. 7A shows a schematic example of the control (top images) and sandwich spatial assay (bottom images) disclosed herein.

FIG. 7B shows an exemplary method of sandwiching two spatially barcoded substrates together.

FIGS. 8A-8C show representative spatial assay images in fresh frozen control and sandwich control assays (FIG. 8A), and in assays in which two spatially barcoded substrates were sandwiched together (FIGS. 8B-8C).

FIG. 9A shows representative gene expression patterns in each of the two spatially barcoded substrates of the disclosure when the biological sample is a fresh frozen mouse brain tissue.

FIG. 9B shows a comparison of analytes of a fresh frozen mouse sample detected on the tissue substrate compared to the transfer substrate.

FIG. 9C shows a chart of overlap of expression of analytes of a fresh frozen mouse sample between the tissue substrate and the transfer substrate. A value of 1.0 demonstrates identical overlap.

FIG. 10A shows representative gene expression patterns in each of the two gene expression arrays of the disclosure when the biological sample is a formalin-fixed paraffin-embedded (FFPE) mouse brain tissue.

FIG. 10B shows a comparison of analytes of an FFPE mouse sample detected on the tissue substrate compared to the transfer substrate.

FIG. 10C shows a chart of overlap of expression of analytes of an FFPE mouse sample between the tissue substrate and the transfer substrate. A value of 1.0 demonstrates identical overlap.

FIG. 11 depicts results from an experiment comparing a non-sandwich control and a sandwich configuration permeabilization condition.

FIG. 12 depicts a comparison between a non-sandwich control and a sandwich configuration permeabilization condition.

FIG. 13 depicts a spatial clustering analysis and analysis of hippocampal transcript Hpca, comparing non-sandwich control and sandwich configuration permeabilization conditions.

FIG. 14 is a schematic diagram depicting an exemplary sandwiching process between a first substrate comprising a biological sample and a second substrate comprising a spatially barcoded array.

FIG. 15A provides perspective view of an example sample handling apparatus in a closed position.

FIG. 15B provides a perspective view of the example sample handling apparatus 1400 in an open position.

FIG. 16A shows an exemplary sandwiching process where a first substrate and a second substrate are brought into proximity with one another.

FIG. 16B shows a fully formed sandwich configuration creating a chamber formed from one or more spacers, a first substrate, and a second substrate.

FIGS. 17A-17C depict a side view and a top view of an exemplary angled closure workflow for sandwiching a first substrate and a second substrate. FIG. 17A depicts the first substrate angled over (superior to) the second substrate. FIG. 17B shows that as the first substrate lowers, and/or as the second substrate rises, the dropped side of the first substrate may contact the drop of the reagent medium. FIG. 17C depicts a full closure of the sandwich between the first substrate and the second substrate with the spacer contacting both the first substrate and the second substrate.

FIGS. 18A-18E depict an example workflow for an angled sandwich assembly. FIG. 18A shows a substrate 1712 positioned and placed on a base with a side of the substrate supported by a spring. FIG. 18B depicts a drop of reagent medium placed on the substrate.

FIG. 18C shows another substrate 1706 positioned above (superior to) substrate 1712 and at an angle substantially parallel with the base. In FIG. 18D, substrate 1706 is lowered toward the substrate 1712 such that a dropped side of the substrate 1706 contacts the drop first. FIG. 18E depicts a full sandwich closure of the substrate 1706 and the substrate 1712 with the drop of reagent medium positioned between the two sides.

FIG. 19A is a side view of an angled closure workflow.

FIG. 19B is a top view of an angled closure workflow.

### DETAILED DESCRIPTION

### I. Introduction

Disclosed herein are methods, kits, systems, and apparatuses for detecting multiple analytes in a biological sample. In particular, disclosed herein are methods, kits, systems, and apparatuses that utilize two spatially barcoded (e.g., gene expression) arrays. The spatially barcoded arrays are used to detect analytes in a biological sample using a sandwiching process described herein. In particular embodiments, both substrates comprise spatially barcoded capture probes and each capture probe comprises at least a capture domain and a spatial barcode. Use of the two substrates in a sandwiching process described herein increases the amount of spatially barcoded capture probes for capturing analytes from a single location in a biological sample. Thus, the disclosure provides methods, kits, systems, and apparatuses that lead to increased information gathered for spatial analysis.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodriques et al., Science 363 (6434): 1463-1467, 2019; Lec et al., Nat. Protoc. 10 (3): 442-458, 2015; Trejo et al., PLOS ONE 14 (2): c0212031, 2019; Chen et al., Science 348 (6233): aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Some general terminologies that may be used in this disclosure can be found in Section (I) (b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I) (d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I) (d) (ii) (13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

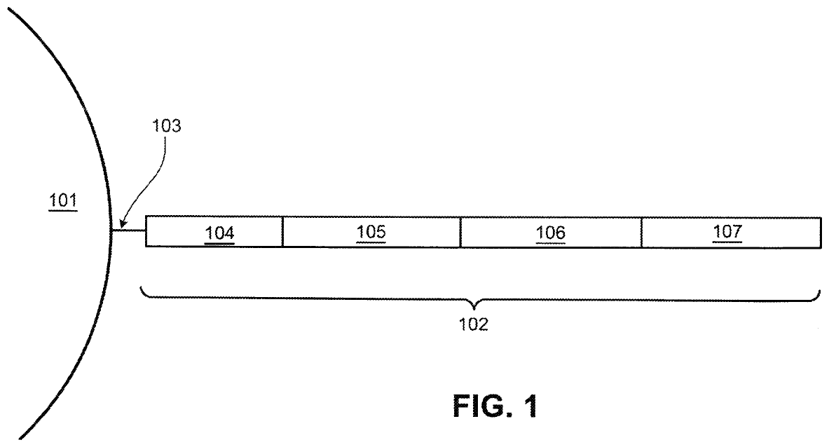

FIG. 1 is a schematic diagram showing an exemplary capture probe, as described herein. As shown, the capture probe 102 is optionally coupled to a feature 101 by a cleavage domain 103, such as a disulfide linker. The capture probe can include a functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte.

The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

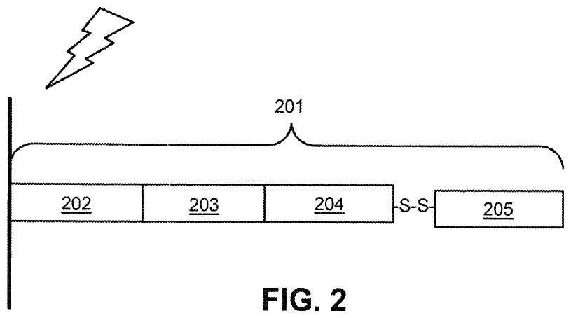

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

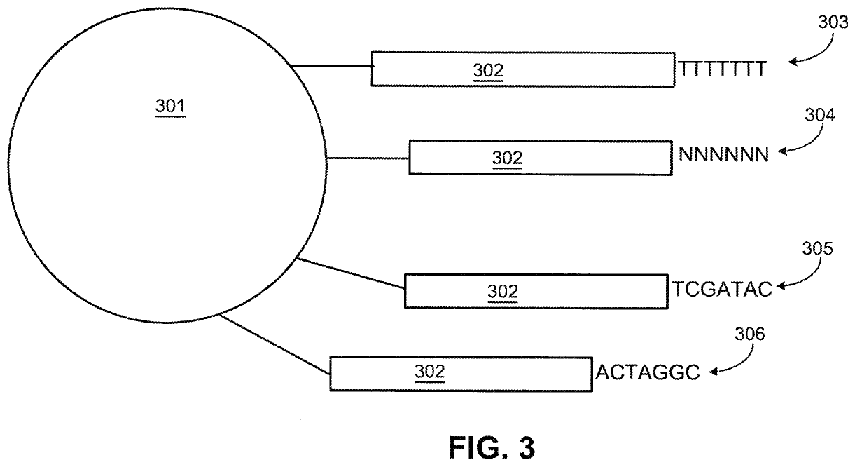

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V (D) J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II) (b) (e.g., subsections (i)-(vi)) of WO 2020/176788 and/or U.S. Patent Application Publication No. 2020/0277663. Generation of capture probes can be achieved by any appropriate method, including those described in Section (II) (d) (ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

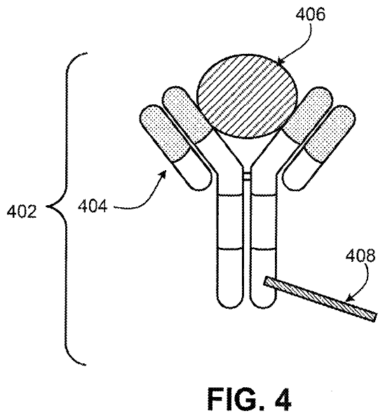

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

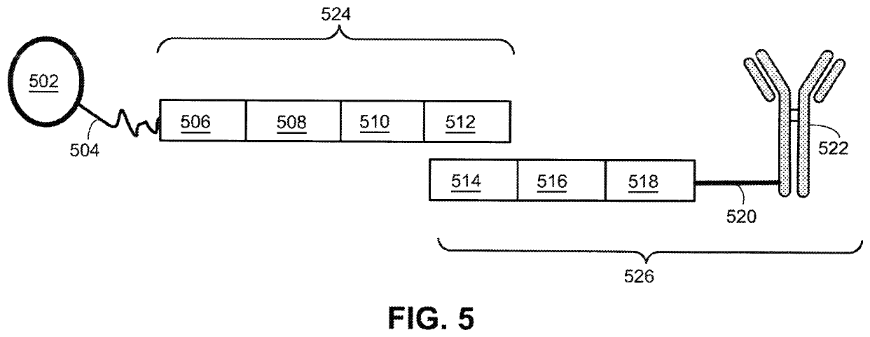

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

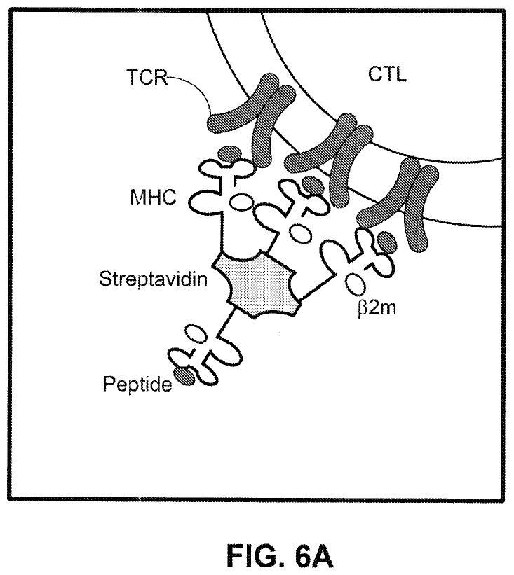

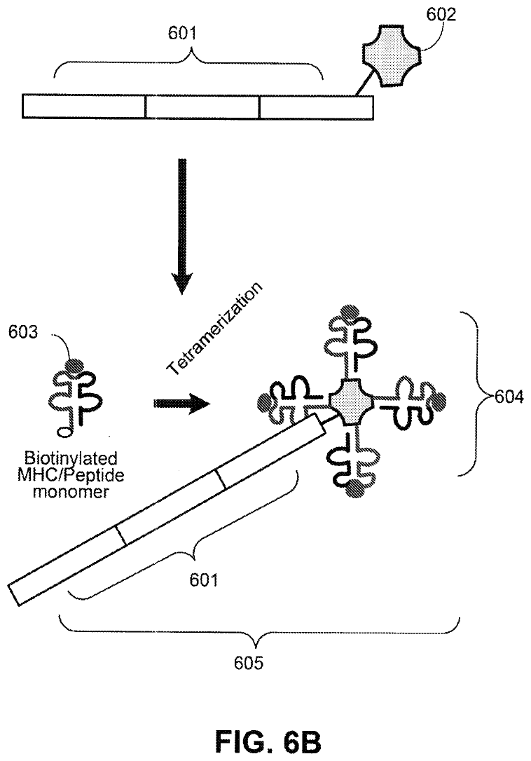

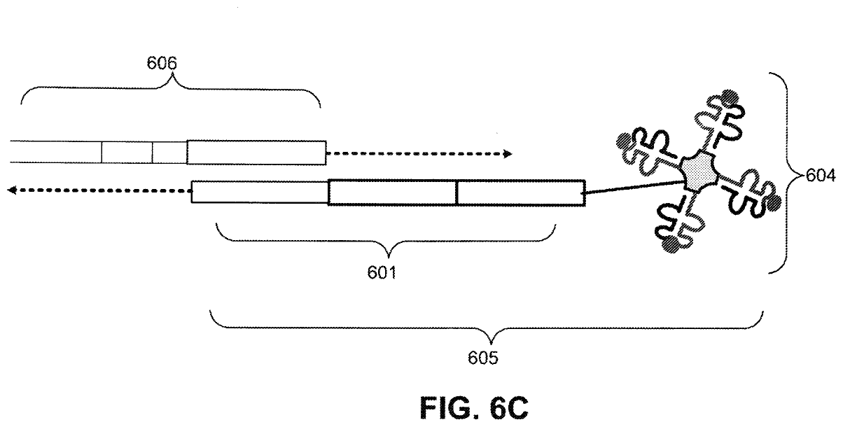

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II) (b) (ix) of WO 2020/176788 and/or Section (II) (b) (viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II) (b) (vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II) (a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II) (g) of WO 2020/176788 and/or U.S. Patent Application Publication No. 2020/0277663. Some quality control measures are described in Section (II) (h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II) (c) of WO 2020/176788 and/or U.S. Patent Application Publication No. 2020/0277663. Exemplary features and geometric attributes of an array can be found in Sections (II) (d) (i), (II) (d) (iii), and (II) (d) (iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II) (c) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45 (14): c128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease. In some embodiments, the endonuclease is an RNAse. In some embodiments, the endonuclease is one of RNase A, RNase C, RNase H, and RNase I. In some embodiments, the endonuclease is RNAse H. In some embodiments, the RNase H is RNase H1 or RNase H2. The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev D, dated October 2020), and/or the Visium Spatial Tissue Optimization Reagent Kits User Guide (e.g., Rev D, dated October 2020). In some embodiments, spatial analysis can be performed using dedicated hardware and/or software, such as any of the systems described in Sections (II) (c) (ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or scalable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

### II. Methods, Compositions, Kits, and Systems for Enhancing Analyte Capture for Spatial Analysis

### (a) Introduction

Disclosed herein are methods, kits, systems, and apparatuses utilizing a spatial array system to assess spatial heterogeneity of analytes in a sample. The methods and systems disclosed herein increase the resolution and number of analytes detected of one or more analytes in a biological sample. The methods and systems here utilize spatial arrays, which include at least two substrates (e.g., gene expression slides) on opposite sides of a biological sample.

Featured herein are methods, kits, systems, and apparatuses for determining the abundance and location of multiple analyte(s) in a biological sample. In some instances, the multiple analytes (e.g., a first analyte and a second analyte) are different analytes. For example, the different analytes can be protein and nucleic acid (e.g., RNA or DNA). Additionally, or in the alternative, the different analytes can be intermediate agent or portion thereof and RNA. The intermediate agent can be a connected probe (e.g., RTL probe) or analyte capture agent (e.g., oligo-conjugated antibody). Additionally, or in the alternative, the different analytes can be RNA and DNA. Additionally, or in the alternative, the different analytes can be intermediate agent and DNA. Additionally, or in the alternative, the different analytes can be targeted capture on one slide and whole transcriptome or DNA on other slide. In some instances, the methods include (a) providing a first substrate and a biological sample mounted thereon, wherein the first substrate comprises a plurality of first capture probes, wherein a first capture probe of the plurality of first capture probes comprises (i) a first spatial barcode and (ii) a first capture domain; (b) aligning a second substrate on the opposite side of the first substrate relative to the biological sample, thereby sandwiching the first substrate, the biological sample, and the second substrate, wherein the second substrate comprises a plurality of second capture probes, wherein a second capture probe of the plurality of second capture probes comprises (i) a second spatial barcode and (ii) a second capture domain; and (c) hybridizing a first analyte of the multiple analytes to the first capture domain and hybridizing a second analyte of the multiple analytes to the second capture domain.

In some instances, the methods further include determining (i) all or a portion of the sequence of the spatial barcode, or a complement thereof, and (ii) all or a portion of the sequence of the one or more analytes captured on the second capture domain, or a complement thereof, and using the sequences of (i) and (ii) to determine the abundance and the location of the analyte in the biological sample.

Also featured herein are methods, kits, systems, and apparatuses that include a first substrate that includes a plurality of capture probes. The capture probes on the first substrate are arranged as an array and include a capture domain described herein (e.g., a capture domain sequence such as a poly(T) (e.g., a poly-thymine sequence or an oligo d (T)) sequence) and a spatial barcode (thus allowing for spatial analysis). The second substrate also includes an array across the substrate or an area of the substrate of capture probes, and the capture probes on the second substrate also include at least a capture domain sequence and spatial barcode. In some instances, the biological sample is provided on the first substrate; after permeabilizing the biological sample analytes are free to disperse from the biological sample. As the analytes passively diffuse, they can be captured by the probes both on the first substrate and the second substrate or on areas of substrates.

Thus, in some instances, this disclosure describes an approach to increase overall capture of analytes by using two gene expression arrays (each comprising capture probes having capture domains and spatial barcodes) in a sandwiching setup. In some instances, this configuration can be applied to barcoded arrays made with printed spots, beads, or microspheres.

(b) Methods and Systems using Two Spatially Barcoded Substrates

The following section provide guidance on the steps of performing the methods to enhance spatial resolution and to detect an analyte in a biological sample using the sandwich configuration disclosed herein. Additional methods of spatial analysis are found in WO 2020/176788, WO 2020/123320, and U.S. Patent Application Publication No. 2020/0277663, each of which is incorporated by reference in its entirety.

As used herein, the first substrate (comprising a plurality of first capture probes, wherein a first capture probe of the plurality of first capture probes comprises (i) a first spatial barcode and (ii) a first capture domain) and the second substrate (comprising a plurality of second capture probes, wherein a second capture probe of the plurality of second capture probes comprises (i) a second spatial barcode and (ii) a second capture domain) may be referred to herein as “spatially barcoded substrates”. The plurality of capture probes on a spatially barcoded substrate may be ordered as an array of capture probes. The capture probes of the array may be ordered into features. Capture probes of a feature of the array may comprise a particular spatial barcode (e.g., a feature-specific spatial barcode), whereas capture probes of a different feature of the array may comprise a different spatial barcode (e.g., a different feature-specific spatial barcode). Accordingly, a spatially barcoded substrate may be referred to herein as a spatially barcoded array or a substrate comprising an array of capture probes. Spatially barcoded substrates with capture domains configured to hybridize to mRNA analytes may be referred to herein as “gene expression substrates,” “gene expression slides,” “GEx slides,” or “gene expression arrays”. Unless explicitly indicated, the terms are interchangeable.

### (i) Providing the Two Spatially Barcoded Substrates and Generating the Sandwich Configuration

In some instances, the biological sample is provided on a first substrate (e.g., a first spatially barcoded substrate (e.g., a first gene expression substrate)). In some instances, a biological sample is placed on the first substrate. As used herein, the first substrate is a substrate that allows the biological sample to adhere to the substrate. In some instances, the first substrate is a glass substrate. In some instances, the first substrate includes materials formed from various glasses, substrate s formed from various polymers, hydrogels, layers and/or films, membranes (e.g., porous membranes), flow cells, wafers, plates, or combinations thereof. In some instances, capture probes on the first substrate includes a spatial barcode and a capture domain. In some instances, a capture probe on the first substrate includes a capture domain (e.g., a poly(T) sequence), a unique molecular identifier, a functional sequence such as a primer, a spatial barcode, or combinations thereof. In some instances, the capture domain on the first substrate includes a poly-thymine (e.g., also called poly(T), poly-d (T), or oligo d (T) throughout) sequence that is complementary to a poly-adenylation sequence. In some instances, the capture probes are distributed on the first substrate inferior to the biological sample. In some instances, a capture probe in the plurality is about 20, about 25, about 30, about 35, about 40, about 45, about 50, about 55, about 60, about 65, about 70, about 75, about 80, about 85, about 90, about 95, about 100, or more single-stranded nucleotides in length. In some instances, a capture probe includes one or more non-naturally occurring nucleotides. In some instances, the capture probes are distributed equally on the array of the first substrate. In some instances, the capture probes on the first substrate are adhered directly (e.g., as described herein). In some instances, the capture probes are placed using printed spots (e.g., as described herein).

In some instances, a second substrate (e.g., a second spatially barcoded substrate (e.g., a second gene expression substrate)) is placed superior to the biological sample opposite to the first substrate, creating a sandwich configuration wherein the biological sample and the permeabilization buffer are located in between the two substrates. In some instances, the second substrate is placed below the biological sample, opposite to the first substrate creating a sandwich configuration wherein the biological sample and the permeabilization buffer are located in between the two substrates. In some instances, the second substrate is a glass substrate. In some instances, the second substrate includes various glasses, substrate s formed from various polymers, hydrogels, layers and/or films, membranes (e.g., porous membranes), flow cells, wafers, plates, or combinations thereof. In some instances, the second substrate includes a plurality of probes (e.g., as described herein). In some instances, one or more probes in the plurality includes a spatial barcode and a capture domain. In some instances, a probe on the second substrate includes a capture domain (e.g., a poly(T) sequence), a unique molecular identifier, a functional sequence such as a primer, a spatial barcode, or combinations thereof. For example, a probe on the second substrate can be a probe as described in FIG. 1. In some instances, the probes on the second substrate are adhered directly to the substrate (e.g., as described herein). In some instances, the probes on the second substrate are placed using printed spots (e.g., as described herein).

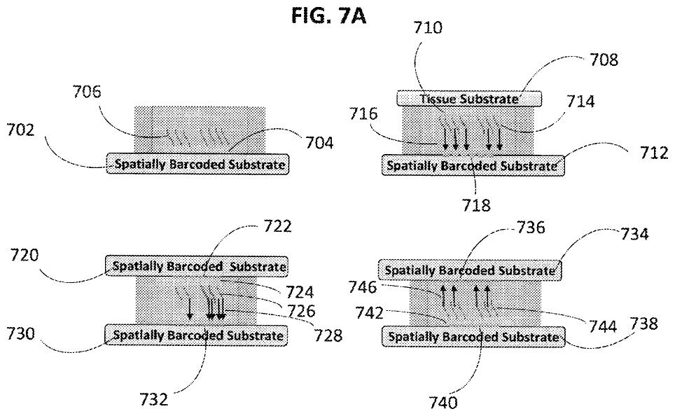

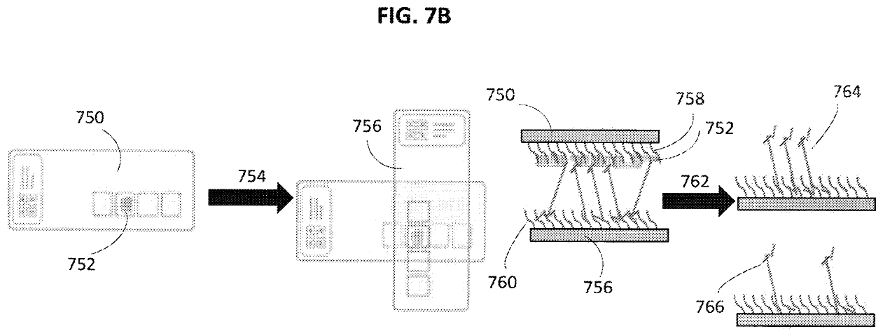

In some instances, the system includes a biological sample sandwiched between two substrates. Referring to FIG. 7A (bottom left image), a biological sample 724 comprising one or more cells is placed on a first substrate 720. It is appreciated that the bottom right image of FIG. 7A represents a situation in which the biological sample is placed on the inferior substrate (compared to the superior substrate in FIG. 7A, bottom left). Thus, the like terms for each can be carried over to each image.

Referring to the bottom left image of FIG. 7A, the first substrate 720 includes an array of probes 722 that include inter alia a capture domain (e.g., a poly-thymine sequence) and a spatial barcode. The array (e.g., plurality) of capture probes can be associated with a whole substrate, parts of a substrate, or defined regions on a substrate. The system includes a permeabilization buffer that is added to the biological sample 724, allowing analytes 726 to diffuse 728 from the biological sample 724. Analytes 726 diffuse from the cell passively in any direction, for example, vertically, horizontally, or laterally, (e.g., arrows represented as 728). In some instances, analytes diffuse from the cell to the array of probes 726 on the first substrate 720. Analytes also migrate to a second substrate 730, which includes a second array (e.g., or i.e., plurality) of capture probes 732. A capture probe or bead (which comprises probes) in the second array includes a capture domain sequence (e.g., a poly-thymine sequence) and a spatial barcode. In some instances, the analyte migrates to the second substrate in a vector, for example, as shown by 728. After migrating to the second substrate, the analyte can be extended, amplified, and sequence using methods disclosed herein. In some instances, still referring to FIG. 7 bottom left image, the first substrate 720 and the second substrate 730 are sandwiched together and form substantially parallel planes. In some instances, the angle of migration of the analyte is measured as an angle from the first substrate 720. In some instances, the angle is 90 degrees relative to the first substrate 720. In some instances, the angle is about 85, about 80, about 75, about 70, about 65, about 60, about 55, about 50, or about 45 degrees relative to the first substrate 720.

In some embodiments, the first substrate 720 and the second substrate 730 are sandwiched together in a sandwiching process. As shown in FIG. 7 bottom left image, during the exemplary sandwiching process, the first substrate is aligned with the second substrate, such that at least a portion of the biological sample is aligned with at least a portion of the array (e.g., aligned in a sandwich configuration). In some embodiments, the first substrate is aligned with the second substrate such that at least a portion of the biological sample is vertically aligned with at least a portion of the array. As shown, the second substrate is in a superior position to the first substrate. In some embodiments, the first substrate may be positioned superior to the second substrate (as depicted in FIG. 7, bottom left image). In some embodiments, the first and second substrates are aligned to maintain a gap or separation distance between the two substrates. When the first and second substrates are aligned, one or more analytes are released from the biological sample and actively or passively migrate to the array for capture. In some embodiments, the migration occurs while the aligned portions of the biological sample and the array are contacted with a reagent medium (e.g., permeabilization buffer). The released one or more analytes may actively or passively migrate across the gap via the reagent medium toward the capture probes or beads on second substrate 730, and be captured by the capture probes 732.

In some embodiments, the separation distance between first and second substrates is maintained between 2 microns and 1 mm (e.g., between 2 microns and 800 microns, between 2 microns and 700 microns, between 2 microns and 600 microns, between 2 microns and 500 microns, between 2 microns and 400 microns, between 2 microns and 300 microns, between 2 microns and 200 microns, between 2 microns and 100 microns, between 2 microns and 25 microns, between 2 microns and 10 microns), measured in a direction orthogonal to the surface of first substrate that supports sample. In some instances, the distance is 2 microns. In some instances, the distance is 2.5 microns. In some instances, the distance is about 2, 3, 4, 5, 6, 7, 8, 9, 10, 11, 12, 13, 14, 15, 16, 17, 18, 19, 20, 21, 22, 23, 24, or 25 microns. In some embodiments, second substrate is placed in direct contact with the sample on the first substrate ensuring no diffusive spatial resolution losses. In some embodiments, the separation distance is measured in a direction orthogonal to a surface of the first substrate that supports the biological sample.

In some embodiments, the first and second substrates are placed in a substrate holder (e.g., an array alignment device) configured to align the biological sample and the array. In some embodiments, the device comprises a sample holder. In some embodiments, the sample holder includes first and second members that comprise first and second retaining mechanisms configured to retain the first and second substrates, respectively. The device can include an alignment mechanism that is connected to at least one of the members and aligns the first and second members. Thus, the devices of the disclosure can advantageously align the first substrate and the second substrate and any samples, barcoded probes, or permeabilization reagents that may be on the surface of the first and second substrates.

In some instances, a sample holder is provided as part of the sandwiching mechanism (e.g., sandwiching apparatus) used in the methods disclosed herein. In some instances, the sample holder includes a first member that includes a first retaining mechanism that retains substrate with sample. In some instances, the sample holder also includes a second member that includes a second retaining mechanism that retains second substrate with a feature array. An alignment mechanism is connected to at least one of first and second members or to both first and second members. During an alignment and contacting procedure, an alignment mechanism functions to align the first and second members, thereby ensuring that sample and feature array are also aligned and brought into contact to facilitate analysis of sample.

In some embodiments, the alignment mechanism can be implemented as a rotating actuator connected to the first and second members. One example of such a rotating actuator is a hinge. In some instances, once a substrate-mounted sample is positioned in the first member and a substrate-mounted feature array is positioned in the second member, rotation of one of the members about the hinge axis aligns members and, and also aligns sample and feature array. The members can be rotated about the hinge axis until the sample and feature array are aligned and in contact. In some instances, the rotating actuator is implemented as a folding member Folding member can be formed from a variety of materials, including compliant materials such as rubber and vinyl, metals and metal alloys, and plastics.

In certain embodiments, the rotating actuator can include at least one arm. In some instances, the rotating actuator can include multiple arms (e.g., 2 or more, 3 or more, 4 or more, or even more). In some instances, sample holder is implemented as a unitary (e.g., i.e., one-piece) device. In some instances, Sample holder can also be implemented as a two-piece device, with first and second members being separate but reproducibly connectable via the alignment mechanism. When the first and second members are brought into proximity, connectors engage with receivers, aligning first and second members, and also aligning sample with the feature array. It should be noted that while connectors are positioned on second member and receivers are positioned on first member, the reverse could also be truc. Moreover, first and second members could each have one or more connectors and one or more receivers.

The first retaining mechanism can be implemented in various ways. In some embodiments, first retaining mechanism can correspond to a recess dimensioned to receive first substrate. Further, a gasket can optionally be positioned within the recess to maintain an interference fit between the edges of the recess and first substrate. In certain embodiments, first retaining mechanism can correspond to one or more members positioned to apply a force to first substrate, in particular, to maintain contact between first substrate and first member. Examples of such members include, but are not limited to, clips, screws and other threaded retaining fasteners, and members that snap-fasten or otherwise engage with first member. The members can apply a force to the sample bearing surface of first substrate and/or to one or more lateral surfaces first substrate.

In general, second retaining mechanism can correspond to any of the different types of retaining mechanisms discussed above in connection with first retaining mechanism. First and second retaining mechanisms and can be different or the same.

In some embodiments, the first member includes a first aperture. The first aperture can be positioned, for example, so that when the first substrate is retained in first member, first aperture is aligned with a sample region (e.g., a region where sample is typically located, or which is designated for placement of sample) on first substrate. Aperture can be positioned so that sample can be viewed from the back surface of first member (e.g., or i.e., the surface opposite to the surface that supports first substrate) through first aperture, and one or more images of sample can be obtained through first aperture.

As described above, a reagent medium can be positioned on a first or second substrate. More generally, however, the first or second substrate may further comprise a reagent medium placed thereon. In certain embodiments, the reagent medium includes a permeabilization reagent (e.g., a solid, liquid, gel, or dried permeabilization reagent). In some embodiments, the reagent medium includes one or more additional components. For example, the additional components can include a hydrogel compound or layer with an embedded permeabilization reagent.

In some embodiments, the second member includes at least one aperture. More generally, the second member can include one or more (e.g., 2 or more, 3 or more, 4 or more, 5 or more, 6 or more, 8 or more, 10 or more, 15 or more, 20 or more, 30 or more, 40 or more, 50 or more, or even more) second apertures. In certain embodiments, the second aperture is aligned with at least a portion of the sample region on substrate when the first and second members and are aligned. A second aperture can used for various purposes. In some embodiments, for example, the feature array and/or the sample can be viewed or imaged through second aperture. Viewing/imaging can be used to adjust the relative positions of the feature array and the sample to improve alignment, for example.

In certain embodiments, one or more bounding surfaces of the second aperture and a back surface of the second substrate (e.g., or i.e., a surface of the second substrate that is opposite to the surface of the second substrate that faces the sample and that supports feature array) cooperate to form a reagent well. A reagent solution (e.g., comprising a permeabilization reagent) added to the reagent well is contained by the bounding surfaces of the second aperture. If the second substrate is formed from a permeable or semi-permeable material, the reagent solution can permeate (e.g., by diffusion) through the back surface of the second substrate and contact the sample.

In some embodiments, the sample holder includes a first adjustment mechanism connected to the first member. The first adjustment mechanism translates the first substrate in at least one direction parallel to the surface of the first substrate that supports the sample. In some embodiments, the first adjustment mechanism translates the first substrate in two directions parallel to the surface of the first substrate.

The first adjustment mechanism can be implemented in various ways. In some embodiments, for example, the first adjustment mechanism includes one or more thumbscrews or linear actuators that can be used to translate the first substrate.

In addition to aligning the first and second members, the alignment mechanism is also configured to maintain a separation between the first and second substrates (and the first and second members) when the substrates (and members) are aligned. For example, the separation can be maintained such that at least a portion of the sample contacts the reagent medium (e.g., the feature array of the reagent medium).

In certain embodiments, the alignment mechanism maintains the first and second substrates in an approximately parallel relationship when the substrates (and the first and second members) are aligned. An included angle between the first and second substrates in such circumstances can be 2 degrees or less (e.g., 1 degree or less, 0.5 degrees or less, 0.25 degrees or less).

In some embodiments, the sample holder can include one or more spacing members that assist in maintaining the spacing and/or approximately parallel arrangement of the first and second substrates. Spacing members can be connected to either or both of the first and second members.

In certain embodiments, the sample holder includes a second adjustment mechanism. The second adjustment mechanism adjusts a distance of the separation between the first and second substrates (e.g., or i.e., in a direction orthogonal to the surface of the first substrate that supports the sample). In certain embodiments, the adjustment mechanism is connected to both members.

The second adjustment mechanism can be implemented in various ways. In some embodiments, the second adjustment mechanism includes one or more thumbscrews or adjustable pins or posts. In certain embodiments, the second adjustment mechanism includes one or more linear actuators. In some embodiments, the second adjustment mechanism includes a swellable or expandable membrane, gasket, or layer positioned between the first and second members.

As a subsequent step in an analytical workflow, after the sample and the feature array have been brought into contact by the sample holder, the sample holder can be introduced into a thermocycler to promote capture of analytes from the sample by the feature array. The sample holder can be inserted directly into a suitable thermocycler for this purpose. Alternatively, in some embodiments, the sample holder can be coupled to a thermocycler adapter and the coupled holder and adapter inserted into a thermocycler. Exemplary devices and exemplary sample holders are described in PCT Patent Application Publication No. WO 2020/123320, which is incorporated by reference in its entirety.

In some instances, the probes on the first and/or second substrate are adhered to beads (e.g., as described herein). In some instances, the probes are placed on the first and/or second substrate using microspheres (e.g., as described herein). In some instances, the beads or microspheres that include probes are associated with, or affixed to, the first and/or second substrate. For example, in some instances capture probe containing beads or microsphere are affixed directly or indirectly to a substrate via surface chemistries, hydrogel, and the like.

In some instances, the diameter of a bead the is adhered to the probe on the second substrate is about 1 μm, about 2 μm, about 3 μm, about 4 μm, about 5 μm, about 6 μm, about 7 μm, about 8 μm, about 9 μm, about 10 μm, about 15 μm, about 20 μm, or more.

The substrate holder is compatible with a variety of different schemes for contacting the aligned portions of the biological sample and array with the reagent medium to promote analyte capture. In some embodiments, the reagent medium is deposited directly on the second substrate (e.g., forming a reagent medium that includes the permeabilization reagent and the feature array), and/or directly on the first substrate. In some embodiments, the reagent medium is deposited on the first and/or second substrate, and then the first and second substrates aligned in the sandwich configuration such that the reagent medium contacts the aligned portions of the biological sample and array. In some embodiments, the reagent medium is introduced into the gap while the first and second substrates are aligned in the sandwich configuration.

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried. Drying methods include, but are not limited to spin coating a thin solution of the reagent and then evaporating a solvent included in the reagent or the reagent itself. Alternatively, in other embodiments, the reagent can be applied in dried form directly onto the first substrate or the second substrate or both. In some embodiments, the coating process can be done in advance of the analytical workflow and the first substrate and the second substrate can be stored pre-coated. Alternatively, the coating process can be done as part of the analytical workflow. In some embodiments, the reagent is a permeabilization reagent. In some embodiments, the reagent is a permeabilization enzyme, a buffer, a detergent, or any combination thereof. In some embodiments, the permeabilization enzyme is pepsin. In some embodiments, the reagent is a dried reagent (e.g., a reagent free from moisture or liquid). In some instances, the substrate that includes the sample (e.g., a histological tissue section) is hydrated. The sample can be hydrated by contacting the sample with a reagent medium, e.g., a buffer that does not include a permeabilization reagent. In some embodiments, the hydration is performed while the first and second substrates are aligned in a sandwich configuration.

In some instances, the aligned portions of the biological sample and the array are in contact with the reagent medium for about 1 minute. In some instances, the aligned portions of the biological sample and the array are in contact with the reagent medium for about 5 minutes. In some instances, the aligned portions of the biological sample and the array are in contact with the reagent medium in the gap for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes. In some instances, the aligned portions of the biological sample and the array are in contact with the reagent medium for about 30 minutes. In some embodiments, following initial contact between sample and a permeabilization agent, the permeabilization agent can be removed from contact with sample (e.g., by opening substrate holder). In some embodiments, the substrate holder is opened before complete permeabilization of sample. For example, in some embodiments, only a portion of sample is permeabilized, and only a portion of the analytes in sample may be captured by feature array. In some instances, the reduced amount of analyte captured and available for detection can be offset by the reduction in lateral diffusion that results from incomplete permeabilization of sample. In general, the spatial resolution of the assay is determined by the extent of analyte diffusion in the transverse direction (e.g., or i.e., orthogonal to the normal direction to the surface of sample). The larger the distance between the sample on the first substrate and the feature array on the second substrate, the greater the extent of diffusion in the transverse direction, and the concomitant loss of resolution. Analytes liberated from a portion of the sample closest to the feature array have a shorter diffusion path, and therefore do not diffuse as far laterally as analytes from portions of the sample farthest from the feature array. As a result, in some instances, incomplete permeabilization of the sample (by reducing the contact interval between the permeabilization agent and the sample) can be used to further increase spatial resolution in the assay. In some embodiments, the substrate holder is opened following complete or substantially complete permeabilization of the sample.

In some instances, the device is configured to control a temperature of the first and second substrates. In some embodiments, the temperature of the first and second members is lowered to a first temperature that is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower). In some embodiments, the device includes a temperature control system (e.g., heating and cooling conducting coils) to control the temperature of the sample holder. Alternatively, in other embodiments, the temperature of the sample holder is controlled externally (e.g., via refrigeration or a hotplate). In a first step, the second member, set to or at the first temperature, contacts the first substrate, and the first member, set to or at the first temperature, contacts the second substrate, thereby lowering the temperature of the first substrate and the second substrate to a second temperature. In some embodiments, the second temperature is equivalent to the first temperature. In some embodiments, the first temperature is lower than room temperature (e.g., 25 degrees Celsius). In some embodiments, the second temperature ranges from about-10 degrees Celsius to about 4 degrees Celsius. In some embodiments, the second temperature is below room temperature (e.g., 25 degrees Celsius) (e.g., 20 degrees Celsius or lower, 15 degrees Celsius or lower, 10 degrees Celsius or lower, 5 degrees Celsius or lower, 4 degrees Celsius or lower, 3 degrees Celsius or lower, 2 degrees Celsius or lower, 1 degree Celsius or lower, 0 degrees Celsius or lower, −1 degrees Celsius or lower, −5 degrees Celsius or lower).

In an exemplary embodiment, the second substrate is contacted with the permeabilization reagent. In some embodiments, the permeabilization reagent is dried. In some embodiments, the permeabilization reagent is a gel or a liquid. Also in the exemplary embodiment, the biological sample is contacted with buffer. Both the first and second substrates are placed at lower temperature to slow down diffusion and permeabilization efficiency. Alternatively, in some embodiments, the sample can be contacted directly with a liquid permeabilization reagent without inducing an unwanted initiation of permeabilization due to the substrates being at the second temperature. In some embodiments, the low temperature slows down or prevents the initiation of permeabilization. In a second step, keeping the sample holder and substrates at a cold temperature (e.g., at the first or second temperatures) continues to slow down or prevent the permeabilization of the sample. In a third step, the sample holder (and consequently the first and second substrates) is heated up to initiate permeabilization. In some embodiments, the sample holder is heated up to a third temperature. In some embodiments, the third temperature is above room temperature (e.g., 25 degrees Celsius) (e.g., 30 degrees Celsius or higher, 35 degrees Celsius or higher, 40 degrees Celsius or higher, 50 degrees Celsius or higher, 60 degrees Celsius or higher). In some embodiments, analytes that are released from the permeabilized tissue of the sample diffuse to the surface of the second substrate and are captured on the array (e.g., barcoded probes) of the second substrate. In a fourth step, the first substrate and the second substrate are separated (e.g., pulled apart) and temperature control is stopped.

In some embodiments, where either the first substrate or substrate second (or both) includes wells, a permeabilization solution can be introduced into some or all of the wells, and then the sample and the feature array can be contacted by closing the sample holder to permeabilize the sample. In certain embodiments, a permeabilization solution can be soaked into a hydrogel film that is applied directly to the sample, and/or soaked into features (e.g., beads) of the array. When the first and second substrates are aligned in the sandwich configuration, the permeabilization solution promotes migration of analytes from the sample to the array.

In certain embodiments, different permeabilization agents or different concentrations of permeabilization agents can be infused into array features (e.g., beads) or into a hydrogel layer as described above. By locally varying the nature of the permeabilization reagent(s), the process of analyte capture from the sample can be spatially adjusted.

In some instances, migration of the analyte from the biological sample to the second substrate is passive (e.g., via diffusion). Alternatively, in certain embodiments, migration of the analyte from the biological sample is performed actively (e.g., electrophoretic, by applying an electric field to promote migration). In some instances, first and second substrates can include a conductive epoxy. Electrical wires from a power supply can connect to the conductive epoxy, thereby allowing a user to apply a current and generate an electric field between the first and second substrates. In some embodiments, electrophoretic migration results in higher analyte capture efficiency and better spatial fidelity of captured analytes (e.g., on a feature array) than random diffusion onto matched substrates without the application of an electric field (e.g., via manual alignment of the two substrates). Exemplary methods of electrophoretic migration, are described in WO 2020/176788, which is hereby incorporated by reference in its entirety.

Loss of spatial resolution can occur when analytes migrate from the sample to the feature array and a component of diffusive migration occurs in the transverse (e.g., lateral) direction, approximately parallel to the surface of the first substrate on which the sample is mounted. To address this loss of resolution, in some embodiments, a permeabilization agent deposited on or infused into a material with anisotropic diffusion can be applied to the sample or to the feature array. The first and second substrates are aligned by the sample holder and brought into contact. A permeabilization layer that includes a permeabilization solution infused into an anisotropic material is positioned on the second substrate.

In some embodiments, the feature array can be constructed atop a hydrogel layer infused with a permeabilization agent. The hydrogel layer can be mounted on the second substrate, or alternatively, the hydrogel layer itself may function as the second substrate. When the first and second substrates are aligned, the permeabilization agent diffuses out of the hydrogel layer and through or around the feature array to reach the sample. Analytes from the sample migrate to the feature array. Direct contact between the feature array and the sample helps to reduce lateral diffusion of the analytes, mitigating spatial resolution loss that would occur if the diffusive path of the analytes was longer.

In some embodiments, the workflow includes provision of the first substrate comprising the biological sample. In some embodiments, the workflow includes, mounting the biological sample onto the first substrate. In some embodiments wherein the biological sample is a tissue sample, the workflow include sectioning of the tissue sample (e.g., cryostat sectioning). In some embodiments, the workflow includes a fixation step. In some instances, the fixation step can include fixation with methanol. In some instances, the fixation step includes formalin (e.g., 2% formalin).

In some embodiments, the biological sample on the first substrate is stained using any of the methods described herein. In some instances, the biological sample is imaged, capturing the stain pattern created during the stain step. In some instances, the biological sample then is destained prior to the sandwiching process.

In some instances, after the sandwiching process the first substrate and the second substrate are separated (e.g., such that they are no longer aligned in a sandwich configuration, also referred to herein as opening the sandwich). In some embodiments, subsequent analysis (e.g., reverse transcription, cDNA synthesis, library preparation, and sequences) can be performed on the captured analytes after the first substrate and the second substrate are separated.

In some embodiments, the process of transferring an analyte from the first substrate to the second substrate is referred to interchangeably herein as a “sandwich process,” “sandwiching process,” or “sandwiching”. The sandwich process is further described in PCT Patent Application Publication No. WO 2020/123320, which is incorporated by reference in its entirety.

In some instances, the permeabilization buffer includes proteinase K, pepsin, collagenase, a detergent, one or more ribonuclease inhibitor, or combinations thereof. In some instances, the detergent is selected from sodium dodecyl sulfate (SDS), polyethylene glycol tert-octylphenyl ether, polysorbate 80, polysorbate 20, N-lauroylsarcosine (or a sodium salt thereof), or combinations thereof. In some instances, the permeabilization buffer comprises a hydrogel.

Prior to analyte capture, in some instances, biological samples can be stained using a wide variety of stains and staining techniques. In some instances, the biological sample is a tissue section on a substrate (e.g., a slide; e.g., a 10 μm biological section section). In some instances, the tissue section is about 5, 6, 7, 8, 9, 10, 11, 12, 13, 14, 15, 16, 17, 18, 19, 20, 21, 22, 23, 24, or 25 μm in thickness.

In some instances, the biological sample is dried after placement onto the first substrate. In some instances, the biological sample is dried at 42° C. In some instances, drying occurs for about 1 hour, about 2, hours, about 3 hours, or until the sections become transparent. In some instances, the biological sample can be dried overnight (e.g., in a desiccator at room temperature).

In some embodiments, a sample can be stained using any number of biological stains, including but not limited to, acridine orange, Bismarck brown, carmine, coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, or safranin. In some instances, the methods disclosed herein include imaging the biological sample. In some instances, imaging the sample occurs prior to deaminating the biological sample. In some instances, the sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation. In some instances, the stain is an H&E stain.

In some embodiments, the biological sample can be stained using a detectable label (e.g., radioisotopes, fluorophores, chemiluminescent compounds, bioluminescent compounds, and dyes) as described elsewhere herein. In some embodiments, a biological sample is stained using only one type of stain or one technique. In some embodiments, staining includes biological staining techniques such as H&E staining. In some embodiments, staining includes identifying analytes using fluorescently-conjugated antibodies. In some embodiments, a biological sample is stained using two or more different types of stains, or two or more different staining techniques. For example, a biological sample can be prepared by staining and imaging using one technique (e.g., H&E staining and brightfield imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in an acid (e.g., HCl). In some embodiments, destaining can include adding HCl to a downstream solution (e.g., permeabilization solution). In some embodiments, destaining can include dissolving an enzyme used in the disclosed methods (e.g., pepsin) in an acid (e.g., HCl) solution. In some embodiments, after destaining hematoxylin with an acid, other reagents can be added to the destaining solution to raise the pH for use in other applications. For example, SDS can be added to an acid destaining solution in order to raise the pH as compared to the acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochcm. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

In some embodiments, immunofluorescence or immunohistochemistry protocols (direct and indirect staining techniques) can be performed as a part of, or in addition to, the exemplary spatial workflows presented herein. For example, tissue sections can be fixed according to methods described herein. The biological sample can be transferred to an array (e.g., capture probe array), wherein analytes (e.g., proteins) are detected using immunofluorescence protocols. For example, the sample can be rehydrated, blocked, and permeabilized (3×SSC, 2% BSA, 0.1% Triton X, 1 U/μl RNAse inhibitor for 10 minutes at 4° C.) before being stained with fluorescent primary antibodies (1:100 in 3×SSC, 2% BSA, 0.1% Triton X, 1 U/μl RNAse inhibitor for 30 minutes at 4° C.). The biological sample can be washed, coverslipped (in glycerol+1 U/μl RNAse inhibitor), imaged (e.g., using a confocal microscope or other apparatus capable of fluorescent detection), washed, and processed according to analyte capture or spatial workflows described herein.

In some instances, a glycerol solution and a cover slip can be added to the sample. In some instances, the glycerol solution can include a counterstain (e.g., DAPI).

As used herein, an antigen retrieval buffer can improve antibody capture in IF/IHC protocols. An exemplary protocol for antigen retrieval can be preheating the antigen retrieval buffer (e.g., to 95° C.), immersing the biological sample in the heated antigen retrieval buffer for a predetermined time, and then removing the biological sample from the antigen retrieval buffer and washing the biological sample.

In some embodiments, optimizing permeabilization can be useful for identifying intracellular analytes. Permeabilization optimization can include selection of permeabilization agents, concentration of permeabilization agents, and permeabilization duration. Tissue permeabilization is discussed elsewhere herein.

In some embodiments, blocking an array and/or a biological sample in preparation of labeling the biological sample decreases nonspecific binding of the antibodies to the array and/or biological sample (decreases background). Some embodiments provide for blocking buffers/blocking solutions that can be applied before and/or during application of the label, wherein the blocking buffer can include a blocking agent, and optionally a surfactant and/or a salt solution. In some embodiments, a blocking agent can be bovine serum albumin (BSA), serum, gelatin (e.g., fish gelatin), milk (e.g., non-fat dry milk), casein, polyethylene glycol (PEG), polyvinyl alcohol (PVA), or polyvinylpyrrolidone (PVP), biotin blocking reagent, a peroxidase blocking reagent, levamisole, Carnoy's solution, glycine, lysine, sodium borohydride, pontamine sky blue, Sudan Black, trypan blue, FITC blocking agent, and/or acetic acid. The blocking buffer/blocking solution can be applied to the array and/or biological sample prior to and/or during labeling (e.g., application of fluorophore-conjugated antibodies) to the biological sample.

In some embodiments of the sandwich configuration disclosed herein, one or more analytes from the biological sample are released from the biological sample and migrate to a substrate comprising an array of capture probes for attachment to the capture probes of the array. In some embodiments, the release and migration of the analytes to the substrate comprising the array of capture probes occurs in a manner that preserves the original spatial context of the analytes in the biological sample. In some embodiments, the biological sample is mounted on a first substrate and the substrate comprising the array of capture probes is a second substrate. In some embodiments, the method is facilitated by a sandwiching process. Sandwiching processes are described in, e.g., US. Patent Application Pub. No. 20210189475, PCT/US2021/036788, and PCT/US2021/050931. In some embodiments, the sandwiching process may be facilitated by a device, sample holder, sample handling apparatus, or system described in, e.g., US. Patent Application Pub. No. 20210189475, PCT/US2021/036788, or PCT/US2021/050931.

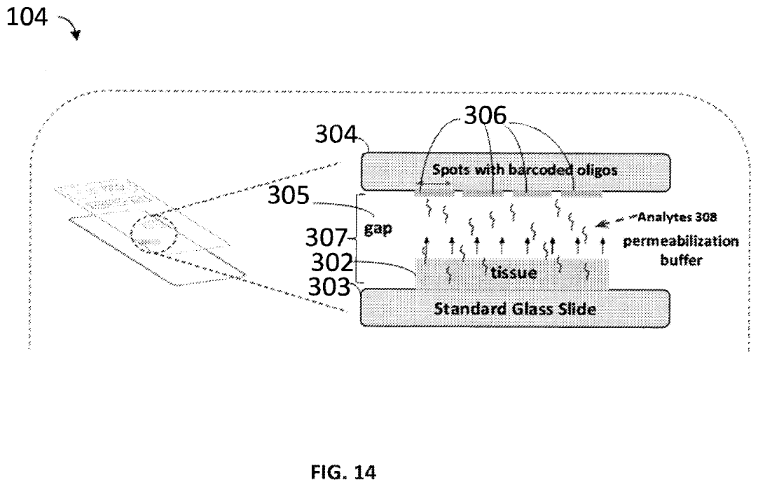

FIG. 14 is a schematic diagram depicting an exemplary sandwiching process 104 between a first substrate comprising a biological sample (e.g., a tissue section 302 on a slide 303) and a second substrate comprising a spatially barcoded array, e.g., a slide 304 that is populated with spatially-barcoded capture probes 306. During the exemplary sandwiching process, the first substrate is aligned with the second substrate, such that at least a portion of the biological sample is aligned with at least a portion of the array (e.g., aligned in a sandwich configuration). As shown, the second substrate (e.g., slide 304) is in a superior position to the first substrate (e.g., slide 303). In some embodiments, the first substrate (e.g., slide 303) may be positioned superior to the second substrate (e.g., slide 304). A reagent medium 305 (e.g., permeabilization solution) within a gap 307 between the first substrate (e.g., slide 303) and the second substrate (e.g., slide 304) creates a permeabilization buffer which permeabilizes or digests the sample 302 and the analytes (e.g., the different analytes described herein, such as, protein, nucleic acid (e.g., RNA or DNA), intermediate agent (e.g., connected probe (e.g., RTL probe) or analyte capture agent (e.g., oligo-conjugated antibody)) or portion thereof, targeted capture, and/or whole transcriptome) 308 of the biological sample 302 may release, actively or passively migrate (e.g., diffuse) across the gap 307 toward the capture probes 306, and bind on the capture probes 306.

After the analytes (e.g., transcripts) 308 bind the capture probes 306, an extension reaction may occur, thereby generating a spatially barcoded library. For example, in the case of mRNA transcripts, reverse transcription may be used to generate a cDNA library associated with a particular spatial barcode. Barcoded cDNA libraries may be mapped back to a specific spot on a capture area of the capture probes 306. This data may be subsequently layered over a high-resolution microscope image of the biological sample, making it possible to visualize the data within the morphology of the tissue in a spatially-resolved manner. In some embodiments, the extension reaction can be performed separately from the sample handling apparatus described herein that is configured to perform the exemplary sandwiching process 104. The sandwich configuration of the sample 302, the first substrate (e.g., slide 303) and the second substrate (e.g., slide 304) may provide advantages over other methods of spatial analysis and/or analyte capture. For example, the sandwich configuration may reduce a burden of users to develop in house tissue sectioning and/or tissue mounting expertise. Further, the sandwich configuration may decouple sample preparation/tissue imaging from the barcoded array (e.g., spatially-barcoded capture probes 306) and enable selection of a particular region of interest of analysis (e.g., for a tissue section larger than the barcoded array). The sandwich configuration also beneficially enables spatial analysis without having to place a biological sample (e.g., tissue section) 302 directly on the second substrate (e.g., slide 304).

In some embodiments, the sandwiching process comprises: mounting the first substrate on a first member of a support device, the first member configured to retain the first substrate; mounting the second substrate on a second member of the support device, the second member configured to retain the second substrate, applying a reagent medium to the first substrate and/or the second substrate, the reagent medium comprising a permeabilization agent, operating an alignment mechanism (also referred to herein as an adjustment mechanism) of the support device to move the first member and/or the second member such that a portion of the biological sample is aligned (e.g., vertically aligned) with a portion of the array of capture probes and within a threshold distance of the array of capture probes, and such that the portion of the biological sample and the capture probe contact the reagent medium, wherein the permeabilization agent releases the analyte from the biological sample.

The sandwiching process methods described above can be implemented using a variety of hardware components. For example, the sandwiching process methods can be implemented using a sample holder (also referred to herein as a support device, a sample handling apparatus, and an array alignment device). In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

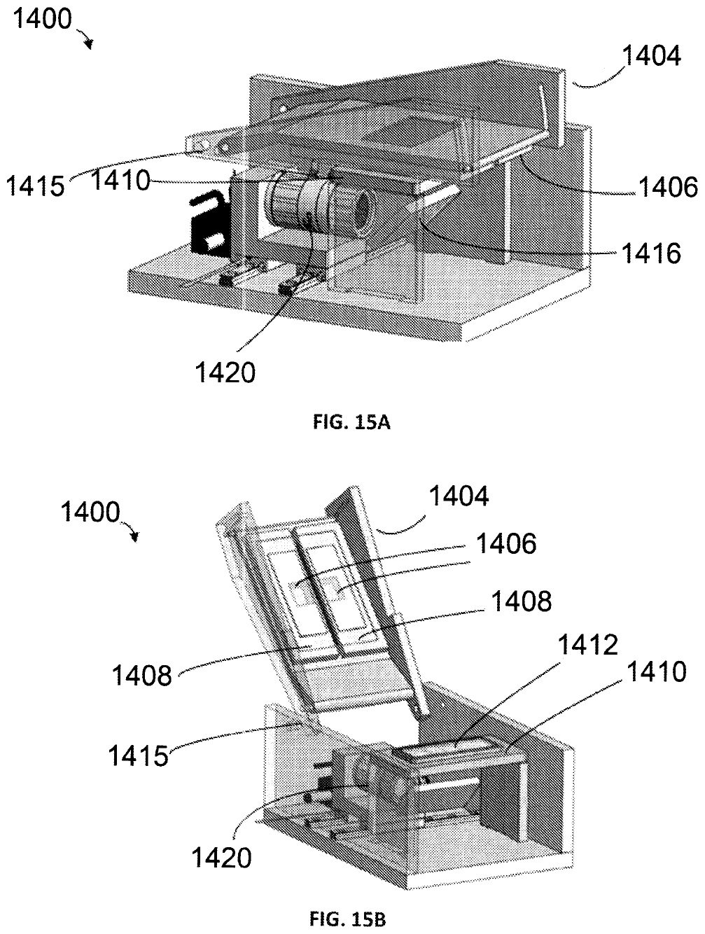

FIG. 15A is a perspective view of an example sample handling apparatus 1400 in a closed position in accordance with some example implementations. As shown, the sample handling apparatus 1400 includes a first member 1404, a second member 1410, optionally an image capture device 1420, a first substrate 1406, optionally a hinge 1415, and optionally a mirror 1416. The hinge 1415 may be configured to allow the first member 1404 to be positioned in an open or closed configuration by opening and/or closing the first member 1404 in a clamshell manner along the hinge 1415.

FIG. 15B is a perspective view of the example sample handling apparatus 1400 in an open position in accordance with some example implementations. As shown, the sample handling apparatus 1400 includes one or more first retaining mechanisms 1408 configured to retain one or more first substrates 1406. In the example of FIG. 15B, the first member 1404 is configured to retain two first substrates 1406, however the first member 1404 may be configured to retain more or fewer first substrates 1406.

In some aspects, when the sample handling apparatus 1400 is in an open position (as in FIG. 15B), the first substrate 1406 and/or the second substrate 1412 may be loaded and positioned within the sample handling apparatus 1400 such as within the first member 1404 and the second member 1410, respectively. As noted, the hinge 1415 may allow the first member 1404 to close over the second member 1410 and form a sandwich configuration (e.g., the sandwich configuration shown in FIG. 14).

In some aspects, after the first member 1404 closes over the second member 1410, an adjustment mechanism (not shown) of the sample handling apparatus 1400 may actuate the first member 1404 and/or the second member 1410 to form the sandwich configuration for the permeabilization step (e.g., bringing the first substrate 1406 and the second substrate 1412 closer to each other and within a threshold distance for the sandwich configuration). The adjustment mechanism may be configured to control a speed, an angle, a force, or the like of the sandwich configuration.

In some embodiments, the biological sample (e.g., sample 302) may be aligned within the first member 1404 (e.g., via the first retaining mechanism 1408) prior to closing the first member 1404 such that a desired region of interest of the sample 302 is aligned with the barcoded array of the second substrate (e.g., the slide 304), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 1406 and/or the second substrate 1412 to maintain a minimum spacing between the first substrate 1406 and the second substrate 1412 during sandwiching. In some aspects, the permeabilization solution (e.g., permeabilization solution 305) may be applied to the first substrate 1406 and/or the second substrate 1412. The first member 1404 may then close over the second member 1410 and form the sandwich configuration. Analytes (e.g., the different analytes described herein, such as, mRNA transcripts) 308 may be captured by the capture probes 306 and may be processed for spatial analysis.

In some embodiments, during the permeabilization step, the image capture device 1420 may capture images of the overlap area (e.g., overlap area 710) between the tissue 302 and the capture probes 306. If more than one first substrates 1406 and/or second substrates 1412 are present within the sample handling apparatus 1400, the image capture device 1420 may be configured to capture one or more images of one or more overlap areas 710. Further details on support devices, sample holders, sample handling apparatuses, or systems for implementing a sandwiching process are described in, e.g., US. Patent Application Pub. No. 20210189475, and PCT/US2021/050931, each of which are incorporated by reference in their entirety.

Analytes within a biological sample may be released through disruption (e.g., permeabilization, digestion, etc.) of the biological sample or may be released without disruption. Various methods of permeabilizing (e.g., any of the permeabilization reagents and/or conditions described herein) a biological sample are described herein, including for example including the use of various detergents, buffers, proteases, and/or nucleases for different periods of time and at various temperatures. Additionally, various methods of delivering fluids (e.g., a buffer, a permeabilization solution) to a biological sample are described herein including the use of a substrate holder (e.g., for sandwich assembly, sandwich configuration, as described herein).

Provided herein are methods for delivering a fluid to a biological sample disposed on an area of a first substrate and an array disposed on a second substrate.

In some embodiments and with reference to FIG. 14, the sandwich configuration described herein between a first substrate comprising a biological sample (e.g., slide 303) and a second substrate comprising a spatially barcoded array (e.g., slide 304 with barcoded capture probes 306) may include a reagent medium (e.g., a liquid reagent medium, e.g., a permeabilization solution 305 or other target molecule release and capture solution) to fill a gap (e.g., gap 307). It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates (e.g., slide 303 and slide 304) during a permeabilization step (e.g., step 104).

In some aspects, it may be possible to reduce or eliminate bubble formation between the slides using a variety of filling methods and/or closing methods.

Workflows described herein may include contacting a drop of the reagent medium (e.g., liquid reagent medium, e.g., a permeabilization solution 305) disposed on a first substrate or a second substrate with at least a portion of the second substrate or first substrate, respectively. In some embodiments, the contacting comprises bringing the two substrates into proximity such that the sample on the first substrate is aligned with the barcode array of capture probes on the second substrate.

In some embodiments, the drop includes permeabilization reagents (e.g., any of the permeabilization reagents described herein). In some embodiments, the rate of permeabilization of the biological sample is modulated by delivering the permeabilization reagents (e.g., a fluid containing permeabilization reagents) at various temperatures.

In the example sandwich maker workflows described herein, the reagent medium (e.g., liquid reagent medium, permeabilization solution 305) may fill a gap (e.g., the gap 307) between a first substrate (e.g., slide 303) and a second substrate (e.g., slide 304 with barcoded capture probes 306) to warrant or enable transfer of target molecules with spatial information. Described herein are examples of filling methods that may suppress bubble formation and suppress undesirable flow of transcripts and/or target molecules or analytes. Robust fluidics in the sandwich making described herein may preserve spatial information by reducing or preventing deflection of molecules as they move from the tissue slide to the capture slide.

FIG. 16A shows an exemplary sandwiching process 3600 where a first substrate (e.g., slide 303), including a biological sample 302 (e.g., a tissue section), and a second substrate (e.g., slide 304 including spatially barcoded capture probes 306) are brought into proximity with one another. As shown in FIG. 16A, a liquid reagent drop (e.g., permeabilization solution 305) is introduced on the second substrate in proximity to the capture probes 306 and in between the biological sample 302 and the second substrate (e.g., slide 304 including spatially barcoded capture probes 306). The permeabilization solution 305 may release analytes that can be captured by the capture probes 306 of the array. As further shown, one or more spacers 3610 may be positioned between the first substrate (e.g., slide 303) and the second substrate (e.g., slide 304 including spatially barcoded capture probes 306). The one or more spacers 3610 may be configured to maintain a separation distance between the first substrate and the second substrate. While the one or more spacers 3610 is shown as disposed on the second substrate, the spacer may additionally or alternatively be disposed on the first substrate.

In some embodiments, the one or more spacers 3610 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 16B shows a fully formed sandwich configuration creating a chamber 3650 formed from the one or more spacers 3610, the first substrate (e.g., the slide 303), and the second substrate (e.g., the slide 304 including spatially barcoded capture probes 306) in accordance with some example implementations. In the example of FIG. 16B, the liquid reagent (e.g., the permeabilization solution 305) fills the volume of the chamber 3650 and may create a permeabilization buffer that allows analytes (e.g., mRNA transcripts and/or other molecules) to diffuse from the biological sample 302 toward the capture probes 306 of the second substrate (e.g., slide 304). In some aspects, flow of the permeabilization buffer may deflect transcripts and/or molecules from the biological sample 302 and may affect diffusive transfer of analytes for spatial analysis. A partially or fully sealed chamber 3650 resulting from the one or more spacers 3610, the first substrate, and the second substrate may reduce or prevent flow from undesirable convective movement of transcripts and/or molecules over the diffusive transfer from the biological sample 302 to the capture probes 306.

In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

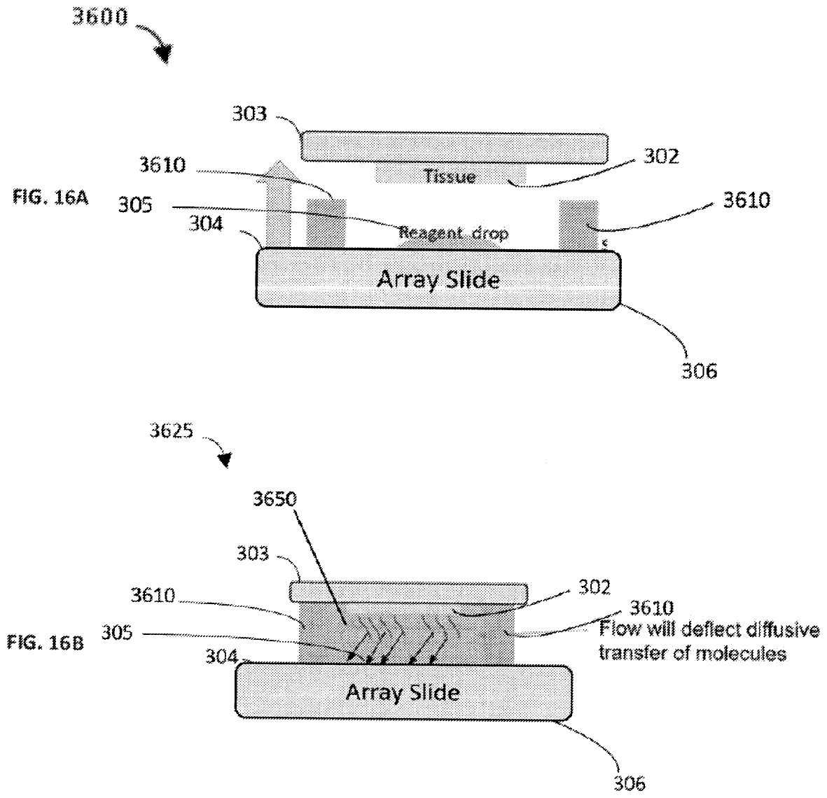

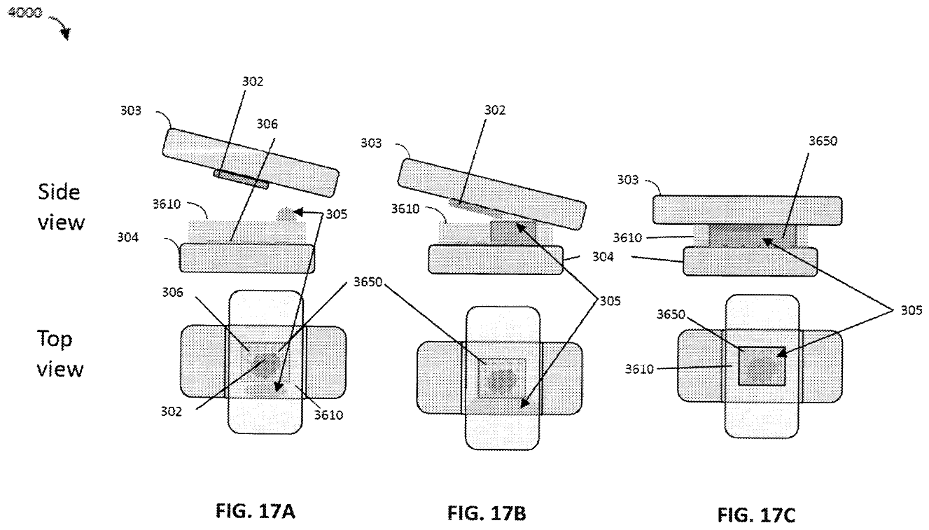

FIGS. 17A-17C depict a side view and a top view of an exemplary angled closure workflow 4000 for sandwiching a first substrate (e.g., slide 303) having a biological sample 302 and a second substrate (e.g., slide 304 having capture probes 306) in accordance with some example implementations.

FIG. 17A depicts the first substrate (e.g., the slide 303 including biological sample 302) angled over (superior to) the second substrate (e.g., slide 304). As shown, a drop of the reagent medium (e.g., permeabilization solution) 305 is located on the spacer 3610 toward the right-hand side of the side view in FIG. 17A. While FIG. 17A depicts the reagent medium on the right hand side of side view, it should be understood that such depiction is not meant to be limiting as to the location of the reagent medium on the spacer.

FIG. 17B shows that as the first substrate lowers, and/or as the second substrate rises, the dropped side of the first substrate (e.g., a side of the slide 303 angled toward the second substrate) may contact the drop of the reagent medium 305. The dropped side of the first substrate may urge the reagent medium 305 toward the opposite direction (e.g., towards an opposite side of the spacer 3610, towards an opposite side of the first substrate relative to the dropped side). For example, in the side view of FIG. 17B the reagent medium 305 may be urged from right to left as the sandwich is formed.

In some embodiments, the first substrate and/or the second substrate are further moved to achieve an approximately parallel arrangement of the first substrate and the second substrate.

FIG. 17C depicts a full closure of the sandwich between the first substrate and the second substrate with the spacer 3610 contacting both the first substrate and the second substrate and maintaining a separation distance and optionally the approximately parallel arrangement between the two substrates. As shown in the top view of FIG. 17C, the spacer 3610 fully encloses and surrounds the biological sample 302 and the capture probes 306, and the spacer 3610 forms the sides of chamber 3650 which holds a volume of the reagent medium 305.

It should be understood that while FIGS. 17A-17C depict the first substrate (e.g., the slide 303 including biological sample 302) angled over (superior to) the second substrate (e.g., slide 304) and the second substrate comprising the spacer 3610, it should be understood that an exemplary angled closure workflow can include the second substrate angled over (superior to) the first substrate and the first substrate comprising the spacer 3610.

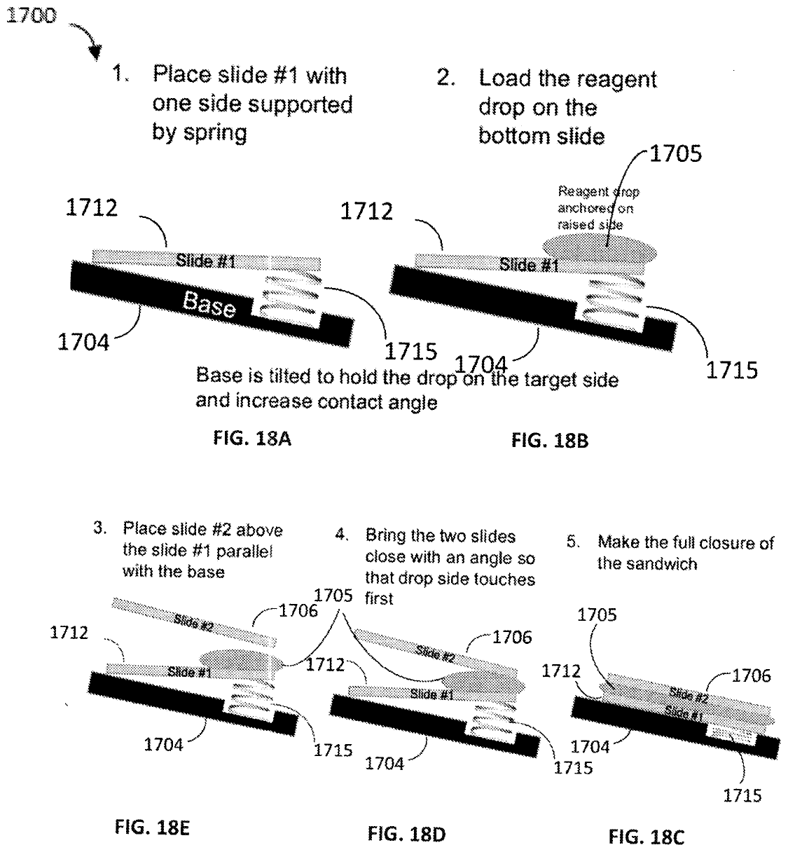

FIGS. 18A-18E depict an example workflow 1700 for an angled sandwich assembly in accordance with some example implementations. As shown in FIG. 18A, a substrate 1712 (e.g., a first substrate such as slide 303 or a second substrate such as slide 304 comprising spatially barcoded capture probes 306) may be positioned and placed on a base 1704 (e.g., a first member or a second member of a sample holder disclosed herein) with a side of the substrate 1712 supported by a spring 1715. The spring 1715 may extend from the base 1704 in a superior direction and may be configured to dispose the substrate 1712 along a plane angled differently than the base 1704. The angle of the substrate 1712 may be such that a drop of reagent medium 1705 (e.g., drop of liquid reagent medium) placed on the surface of the substrate 1712 (e.g., a surface of a spacer attached to the substrate) will not fall off the surface (e.g., due to gravity). The angle may be determined based on a gravitational force versus any surface force to move the drop away from and off the substrate 1712.

FIG. 18B depicts a drop 1705 of reagent medium placed on the substrate 1712. As shown, the drop 1705 is located on the side of the substrate 1712 contacting the spring 1715 and is located in proximity and above (superior to) the spring 1715.

As shown in FIG. 18C, another substrate 1706 may be positioned above (superior to) the substrate 1712 and at an angle substantially parallel with the base 1704. For example, in cases wherein substrate 1712 is a second substrate disclosed herein (e.g., slide 304 comprising spatially barcoded capture probes), substrate 1706 may be a first substrate disclosed herein (e.g., slide 303). In cases wherein substrate 1712 is a first substrate disclosed herein (e.g., slide 303), substrate 1706 may be a second substrate (e.g., slide 304 comprising spatially barcoded capture probes).

In some cases, another base (not shown) supporting substrate 1706 (e.g., a first member or a second member of a sample holder disclosed herein) may be configured to retain substrate 1706 at the angle substantially parallel to the base 1704.

As shown in FIG. 18D, substrate 1706 may be lowered toward the substrate 1712 such that a dropped side of the substrate 1706 contacts the drop 1705 first. In some aspects, the dropped side of the substrate 1706 may urge the drop 1705 toward the opposite side of the substrate 1706. In some embodiments, the substrate 1712 may be moved upward toward the substrate 1706 to accomplish the contacting of the dropped side of the substrate 1706 with the drop 1705.

FIG. 18E depicts a full sandwich closure of the substrate 1706 and the substrate 1712 with the drop of reagent medium 1705 positioned between the two sides. In some aspects and as shown, as the substrate 1706 is lowered onto the drop 1705 and toward the substrate 1712 (and/or as the substrate 1712 is raised up toward the substrate 1706), the spring 1715 may compress and the substrate 1712 may lower to the base 1704 and become substantially parallel with the substrate 1706.

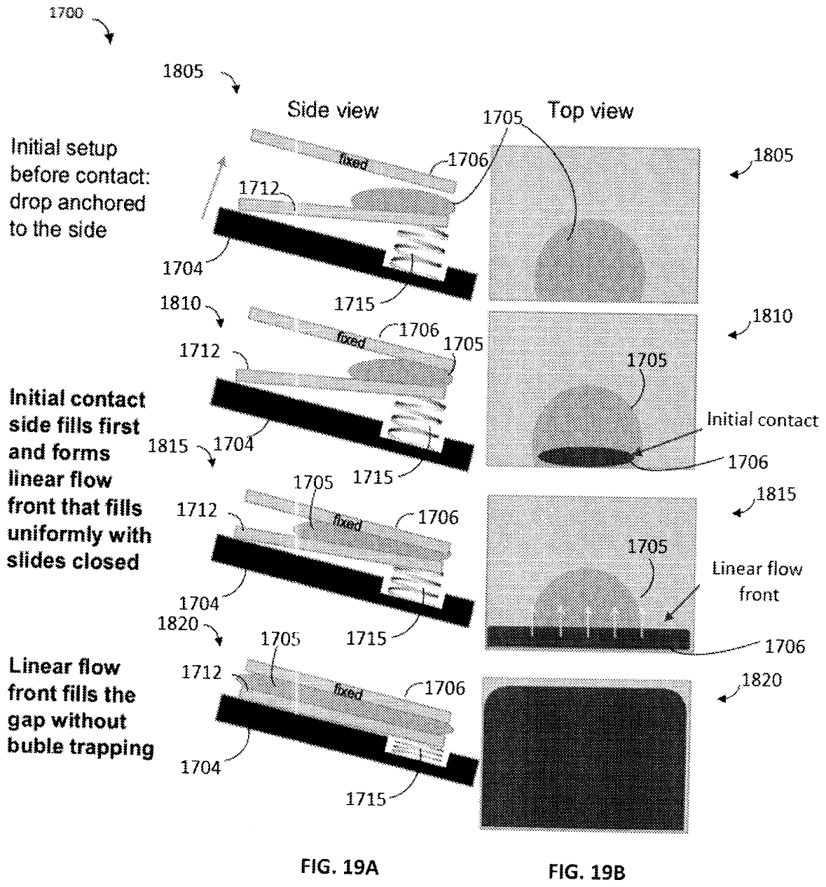

FIG. 19A is a side view of the angled closure workflow 1700 in accordance with some example implementations. FIG. 19B is a top view of the angled closure workflow 1700 in accordance with some example implementations. As shown at 1805 and in accordance with FIGS. 18C-D, the drop of reagent medium 1705 is positioned to the side of the substrate 1712 contacting the spring 1715.

At step 1810, the dropped side of the angled substrate 1706 contacts the drop of reagent medium 1705 first. The contact of the substrate 1706 with the drop of reagent medium 1705 may form a linear or low curvature flow front that fills uniformly with the slides closed.

At step 1815, the substrate 1706 is further lowered toward the substrate 1712 (or the substrate 1712 is raised up toward the substrate 1706) and the dropped side of the substrate 1706 may contact and may urge the liquid reagent toward the side opposite the dropped side and creating a linear or low curvature flow front that may prevent or reduce bubble trapping between the slides. As further shown, the spring 1715 may begin to compress as the substrate 1706 is lowered.

At step 1820, the drop of reagent medium 1705 fills the gap (e.g., the gap 307) between the substrate 1706 and the substrate 1712. The linear flow front of the liquid reagent may form by squeezing the drop 1705 volume along the contact side of the substrate 1712 and/or the substrate 1706. Additionally, capillary flow may also contribute to filling the gap area. As further shown in step 1820, the spring 1715 may be fully compressed such that the substrate 1706, the substrate 1712, and the base 1704 are substantially parallel to each other.

In some aspects, an angled closure workflow disclosed herein (e.g., FIGS. 17A-17C, 18A-18E, 19A-19B) may be performed by a sample handling apparatus (e.g., as described in PCT/US2021/050931, which is hereby incorporated by reference in its entirety.

Further details on angled closure workflows, and devices and systems for implementing an angled closure workflow, are described in PCT/US2021/036788 and PCT/US2021/050931, which are hereby incorporated by reference in their entirety. Additional configurations for reducing or eliminating bubble formation, and/or for reducing unwanted fluid flow, are described in PCT/US2021/036788, which is hereby incorporated by reference in its entirety.

### (ii) Preparation of Biological Sample for Analyte Capture

The disclosure provides methods, kits, substrates, and apparatuses that examine spatial analyte expression in multiple tissue types. In some instances, the methods provided herein are performed on an FFPE sample. In some instances, the methods provided herein are performed on a fresh frozen sample.

In some instances (e.g., in an FFPE sample), the biological sample is deparaffinized. Deparaffinization can be achieved using any method known in the art. For example, in some instances, the biological samples is treated with a series of washes that include xylene and various concentrations of ethanol. In some instances, methods of deparaffinization include treatment of xylene (e.g., three washes at 5 minutes each). In some instances, the methods further include treatment with ethanol (e.g., 100% ethanol, two washes 10 minutes each; 95% ethanol, two washes 10 minutes each; 70% ethanol, two washes 10 minutes each; 50% ethanol, two washes 10 minutes each). In some instances, after ethanol washes, the biological sample can be washed with deionized water (e.g., two washes for 5 minutes each). It is appreciated that one skilled in the art can adjust these methods to optimize deparaffinization. In some instances, the biological sample is decrosslinked. In some instances, the biological sample is decrosslinked in a solution containing TE buffer (comprising Tris and EDTA). In some instances, the TE buffer is basic (e.g., at a pH of about 9). In some instances, decrosslinking occurs at about 50° C. to about 80° C. In some instances, decrosslinking occurs at about 70° C. In some instances, decrosslinking occurs for about 1 hour at 70° C. Just prior to decrosslinking, the biological sample can be treated with an acid (e.g., 0.1M HCl for about 1 minute). After the decrosslinking step, the biological sample can be washed (e.g., with 1×PBST).

In some instances, the methods of preparing a biological sample for analyte capture include steps of equilibrating and blocking the biological sample. In some instances, equilibrating is performed using a pre-hybridization (pre-Hyb) buffer. In some instances, the pre-Hyb buffer is RNase-free. In some instances, the pre-Hyb buffer contains no bovine serum albumin (BSA), solutions like Denhardt's, or other potentially nuclease-contaminated biological materials.

In some instances, the equilibrating step is performed multiple times (e.g., 2 times at 5 minutes each; 3 times at 5 minutes each). In some instances, the biological sample is blocked with a blocking buffer. In some instances, the blocking buffer includes a carrier such as tRNA, for example yeast tRNA such as from brewer's yeast (e.g., at a final concentration of 10-20 μg/mL). In some instances, blocking can be performed for 5, 10, 15, 20, 25, or 30 minutes.

Any of the foregoing steps can be optimized for performance. For example, one can vary the temperature. In some instances, the pre-hybridization methods are performed at room temperature. In some instances, the pre-hybridization methods are performed at 4° C. (in some instances, varying the timeframes provided herein).

(iii) Simultaneous Capture on the First Substrate and Second Substrate

In some instances, the methods of preparing a biological sample for analyte capture include permeabilizing the sample. In some instances, the biological sample is permeabilized using a phosphate buffer. In some instances, the phosphate buffer is PBS (e.g., 1×PBS). In some instances, the phosphate buffer is PBST (e.g., 1×PBST). In some instances, the permeabilization step is performed multiple times (e.g., 3 times at 5 minutes each).

In some instances, a permeabilization buffer (e.g., any permeabilization buffer described herein) is added to the biological sample. Permeabilization solutions can include, by way of example only, enzymes (e.g., proteinase K, pepsin, and collagenase), detergents (e.g., N-lauroylsarcosine or a sodium salt thereof, sodium dodecyl sulfate (SDS), polyethylene glycol tert-octylphenyl ether, polysorbate 80, and polysorbate 20), ribonuclease inhibitors, buffers optimized for electrophoresis, buffers optimized for permeabilization, buffers optimized for hybridization, or combinations thereof. The permeabilization buffer releases the analyte from the sample, allowing it to diffuse from the sample. In some instances, the biological sample can be treated with a proteinase. In some instances, the proteinase is proteinase K.

In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, the biological sample is permeabilized prior to capture of the analytes on either the first substrate or the second substrate (or both).

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K, and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

In some embodiments, the methods provided herein include a permeabilizing step in order to release the analyte. In some embodiments, permeabilization occurs using a protease. In some embodiments, the protease is an endopeptidase. Endopeptidases that can be used include but are not limited to trypsin, chymotrypsin, elastase, thermolysin, pepsin, clostripan, glutamyl endopeptidase (GluC), ArgC, peptidyl-asp endopeptidase (ApsN), endopeptidase LysC and endopeptidase LysN. In some embodiments, the endopeptidase is pepsin. In some embodiments, methods provided herein include permeabilization of the biological sample such that the capture probe can more easily bind to the analyte (e.g., or i.e., compared to no permeabilization).

In some instances, the permeabilization step includes application of a permeabilization buffer to the biological sample. In some instances, the permeabilization buffer includes a buffer (e.g., Tris pH 7.5), MgCl2, sarkosyl detergent (e.g., sodium lauroyl sarcosinate), enzyme (e.g., proteinase K), and nuclease free water. In some instances, the permeabilization step is performed at 37° C. In some instances, the permeabilization step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 40 minutes.

In some embodiments, the analyte is released using an endoribonuclease. In some embodiments, the endoribonuclease is an RNase. The RNase can be RNase H, RNase A, RNase C, or RNase I. In some embodiments, the RNase H is RNase H1, RNase H2, or RNase H1, or RNase H2.

In some instances, the releasing step is performed using a releasing buffer. In some instances, the release buffer includes one or more of a buffer (e.g., Tris pH 7.5), enzyme (e.g., RNAse H) and nuclease-free water. In some instances, the releasing step is performed at 37° C. In some instances, the releasing step is performed for about 20 minutes to 2 hours (e.g., about 20 minutes, about 30 minutes, about 40 minutes, about 50 minutes, about 1 hour, about 1.5 hours, or about 2 hours). In some instances, the releasing step is performed for about 30 minutes.

In some instances, the releasing step occurs after the permeabilization step. In some instances, the releasing step occurs at the same time as the permeabilization step (e.g., in the same buffer).

In some embodiments, the reagent medium (e.g., the permeabilization buffer) comprises one or more of sodium dodecyl sulfate (SDS), proteinase K, pepsin, N-lauroylsarcosine or a sodium salt thereof, RNAse, and a sodium salt thereof.

In some instances, a hydrogel is used to enhance spatial resolution. In some embodiments, a biological sample (e.g., tissue section) is embedded in a hydrogel. In some embodiments, hydrogel subunits are infused into the biological sample, and polymerization of the hydrogel is initiated by an external or internal stimulus. A “hydrogel” as described herein can include a cross-linked 3D network of hydrophilic polymer chains. A “hydrogel subunit” can be a hydrophilic monomer, a molecular precursor, or a polymer that can be polymerized (e.g., cross-linked) to form a three-dimensional (3D) hydrogel network. Additional disclosure of hydrogels is found in WO 2020/176788 and U.S. Patent Application Publication No. 2020/0277663, each of which is incorporated by reference in its entirety.

In some instances, analytes migrate through the biological sample. In some instances, analytes migrate from the biological sample to the first and/or second probe. In some instances, migration disclosed herein is passive migration. As some migration is passive, analytes (e.g., mRNA) can migrate in any direction. In some instances, probes on the first substrate capture analytes that migrate passively. In some instances, probes on the second substrate capture analytes that migrate passively.

In some embodiments, after a certain period of time (e.g., about 5 minutes to about 10 hours, about 5 minutes to about 5 hours, about 5 minutes to about 1 hour, about 5 minutes to about 45 minutes, about 5 minutes to about 30 minutes, about 5 minutes to about 15 minutes, about 15 minutes to about 10 hours, about 15 minutes to about 5 hours, about 15 minutes to about 1 hour, about 15 minutes to about 45 minutes, about 15 minutes to about 30 minutes, about 30 minutes to about 10 hours, about 30 minutes to about 5 hours, about 30 minutes to about 1 hour, about 30 minutes to about 45 minutes, about 45 minutes to about 10 hours, about 45 minutes to about 5 hours, about 45 minutes to about 1 hour, about 1 hour to about 10 hours, about 1 hour to about 5 hours, about 1 hour to about 1.5 hours, about 1.5 hours to about 10 hours, about 1.5 hours to about 5 hours, about 1.5 hours to about 2 hours, about 2 hours to about 10 hours, about 2 hours to about 5 hours, about 2 hours to about 3 hours, about 2.5 hours to about 10 hours, about 2.5 hours to about 5 hours, about 2.5 hours to about 3 hours, about 3 hours to about 10 hours, about 3 hours to about 5 hours, about 4 hours to about 10 hours, about 4 hours to about 5 hours, about 5 hours to about 10 hours, about 6 hours to about 10 hours, about 7 hours to about 10 hours, about 8 hours to about 10 hours, or about 9 hours to about 10 hours), one or more analytes have passively migrated and been captured by the capture probe(s) on the first substrate. In some embodiments, after a certain period of time as previously listed, one or more analytes have passively migrated and been captured by the capture probe(s) on the second substrate. In some instances, at least about 80%, or at least about 90% of all analytes are captured by probes on the second substrate. It is contemplated that while a portion of the analytes, for example those in close proximity to the first substrate capture probes, will passively migrate and be captured on the first substrate, whereas the majority of the analytes, those that are not in close proximity to the first substrate, will passively migrate to the second substrate and be captured by the barcoded capture probes.

In some embodiments, there is a gap (e.g., a space) between the first substrate/biological sample and the second substrate that is filled with one or more solutions. In some embodiments, the one or more solutions between the first substrate/biological sample and the second substrate can include a permeabilization buffer (e.g., any of the permeabilization buffers described herein). In some embodiments, the one or more solutions can include a buffer that can maintain the pH at a relatively constant value. In some instances, the gap between the biological sample and the second substrate with the second set of capture probes is about 0.5 μm, about 1.0 μm, about 1.5 μm, about 2.0 μm, about 2.5 μm, about 3.0 μm, about 3.5 μm, about 4.0 μm, about 4.5 μm, about 5.0 μm, or more. In some instances, the distance between the biological sample and the second set of capture probes is about 2.5 μm.

In some embodiments, the method described herein further comprises separating the first substrate and the second substrates after the capture of the analytes. In some embodiments, the first substrate and the second substrate are de-aligned.

In some instances, compared to a sandwich configuration that does not include probes on the first substrate, the spatial resolution of analytes from a biological sample is increased. Thus, in some instances, the resolution of detection of analytes is increased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 1.5 fold, about 2-fold, about 3-fold, about 4-fold, about 5-fold, about 10-fold, about 15-fold, about 20-fold, or more compared to a sandwich configuration that does not include probes on the first substrate.

In some instances, the resolution of detection of an analyte can be determined by measuring the width of the captured analyte zone. In some instances, increased resolution of detection of the analyte corresponds to decreased width of the captured analyte zone. In some instances, the width of the captured analyte zone is decreased by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100% or more compared to a sandwich configuration that does not include probes on the first substrate.

### (iv) Analyte Analysis on the First Substrate and Second Substrate

In some embodiments, as disclosed herein, the analytes from the biological sample hybridize to the capture probe on both the first substrate and the second substrate. As depicted in FIG. 7 bottom left image, one or more analytes from the biological sample may be captured by capture probes 722 of the first substrate 720, and one or more analytes from the biological sample may be captured by second capture probes 732 of the second substrate 730. After capture of the analytes on the first substrate or the second substrate, in some instances, the methods provided herein include determining the spatial expression (e.g., abundance and location) of the analytes in the biological sample using the captured analytes on the first substrate and/or the second substrate. For example, the analytes may be captured by the first capture probes or second capture probes in a manner that retains spatial context of the analytes in the biological sample. Generally, after hybridization of the analyte to the capture probe on the first substrate and/or the second substrate, the capture probe is extended at the 3′ end and a copy of the additional capture probe components (e.g., the spatial barcode) of the capture probe is synthesized. In some embodiments, reverse transcription (RT) reagents can be added to the permeabilized biological samples. Incubation with the RT reagents can produce spatially-barcoded full-length cDNA from the captured analytes (e.g., polyadenylated mRNA). Second strand reagents (e.g., second strand primers, enzymes) can additionally be added to initiate second strand synthesis.

In some embodiments, one or more analytes migrate through a solution and specifically bind (e.g., hybridize) to the capture domains on the capture probes of the first substrate and/or the second substrate. In some embodiments, one or more mRNAs migrate through a solution that includes a permeabilization buffer and bind (e.g., hybridize) to the capture domains on the capture probes on the first substrate and/or the second substrate. In some embodiments, the sequence of all or a portion of the capture probes (e.g., the spatial barcode or a portion thereof) or a complement thereof, on the first substrate and/or the second substrate and all or a portion of the sequence of the corresponding captured analyte (or a complement thereof) are determined. In some embodiments, the location of the one or more analytes in the biological sample are determined based on all or a portion of the sequence of the capture probes (e.g., the spatial barcode or a portion thereof) on the array, or a complement thereof, and all or a portion of the sequence of the corresponding captured one or more analytes, or a complement thereof. In some embodiments, determining the location of the one or more analytes in the biological sample includes determining all or a portion of the sequence of the capture probes (e.g., the spatial barcode, or a portion thereof) on the array, or a complement thereof, and all or a portion of the sequence of the corresponding captured one or more analytes, or complement thereof, and correlating such sequence information to an image of the biological sample. Some embodiments of these methods further include obtaining an image of the biological sample.

In some embodiments, after an analyte from the sample has hybridized or otherwise been associated with a capture probe on the first substrate and/or the second substrate according to any of the methods described above in connection with the general spatial cell-based analytical methodology, the barcoded constructs that result from hybridization/association are analyzed.

In some embodiments, after contacting a biological sample with the first substrate and/or the second substrate that includes capture probes, a removal step can optionally be performed to remove all or a portion of the biological sample from the substrate. In some embodiments, the removal step includes enzymatic and/or chemical degradation of cells of the biological sample. For example, the removal step can include treating the biological sample with an enzyme (e.g., a proteinase, e.g., proteinase K) to remove at least a portion of the biological sample from the substrate. In some embodiments, the removal step can include ablation of the tissue (e.g., laser ablation).

In some embodiments, provided herein are methods for spatially detecting an analyte (e.g., detecting the location of an analyte, e.g., a biological analyte) from a biological sample (e.g., present in a biological sample), the method comprising: (a) optionally staining and/or imaging a biological sample on a substrate; (b) permeabilizing (e.g., providing a solution comprising a permeabilization reagent to) the biological sample on the substrate; (c) contacting the biological sample with the first substrate and/or the second substrate comprising a plurality of capture probes, wherein a capture probe of the plurality captures the biological analyte; and (d) analyzing the captured biological analyte, thereby spatially detecting the biological analyte; wherein the biological sample is fully or partially removed from the substrate.

In some embodiments, a biological sample is not removed from the substrate. For example, the biological sample is not removed from the substrate prior to releasing a capture probe (e.g., a capture probe bound to an analyte) from the substrate. In some embodiments, such releasing comprises cleavage of the capture probe from the substrate (e.g., via a cleavage domain). In some embodiments, such releasing does not comprise releasing the capture probe from the substrate (e.g., a copy of the capture probe bound to an analyte can be made and the copy can be released from the substrate, e.g., via denaturation). In some embodiments, the biological sample is not removed from the substrate prior to analysis of an analyte bound to a capture probe after it is released from the substrate. In some embodiments, the biological sample remains on the substrate during removal of a capture probe from the substrate and/or analysis of an analyte bound to the capture probe after it is released from the substrate. In some embodiments, the biological sample remains on the substrate during removal (e.g., via denaturation) of a copy of the capture probe (e.g., complement). In some embodiments, analysis of an analyte bound to capture probe from the substrate can be performed without subjecting the biological sample to enzymatic and/or chemical degradation of the cells (e.g., permeabilized cells) or ablation of the tissue (e.g., laser ablation).

In some embodiments, at least a portion of the biological sample is not removed from the substrate. For example, a portion of the biological sample can remain on the substrate prior to releasing a capture probe (e.g., a capture prove bound to an analyte) from the substrate and/or analyzing an analyte bound to a capture probe released from the substrate. In some embodiments, at least a portion of the biological sample is not subjected to enzymatic and/or chemical degradation of the cells (e.g., permeabilized cells) or ablation of the tissue (e.g., laser ablation) prior to analysis of an analyte bound to a capture probe from the substrate.

In some embodiments, provided herein are methods for spatially detecting an analyte (e.g., detecting the location of an analyte, e.g., a biological analyte) from a biological sample (e.g., present in a biological sample) that include: (a) optionally staining and/or imaging a biological sample on a substrate; (b) permeabilizing (e.g., providing a solution comprising a permeabilization reagent to) the biological sample on the substrate; (c) contacting the biological sample with a first substrate and/or a second substrate comprising a plurality of capture probes, wherein a capture probe of the plurality captures the biological analyte; and (d) analyzing the captured biological analyte, thereby spatially detecting the biological analyte; where the biological sample is not removed from the substrate.

In some embodiments, provided herein are methods for spatially detecting a biological analyte of interest from a biological sample that include: (a) staining and imaging a biological sample on a substrate; (b) providing a solution comprising a permeabilization reagent to the biological sample on the substrate; (c) contacting the biological sample with an array on a substrate, wherein the array comprises one or more capture probe pluralities thereby allowing the one or more pluralities of capture probes to capture the biological analyte of interest; and (d) analyzing the captured biological analyte, thereby spatially detecting the biological analyte of interest; where the biological sample is not removed from the substrate.

In some embodiments, the method further includes subjecting at least a portion of the biological sample to spatial omics analysis (e.g., spatial transcriptomic analysis). In some embodiments, the method further includes subjecting a region of interest in the biological sample to spatial omics analysis (e.g., spatial transcriptomic analysis). In some embodiments, one or more of the capture probes includes a capture domain. In some embodiments, one or more of the capture probes comprises a unique molecular identifier (UMI). In some embodiments, one or more of the capture probes comprises a cleavage domain. In some embodiments, the cleavage domain comprises a sequence recognized and cleaved by a uracil-DNA glycosylase, apurinic/apyrimidinic (AP) endonuclease (APE1), U uracil-specific excision reagent (USER), and/or an endonuclease VIII. In some embodiments, one or more capture probes do not comprise a cleavage domain and is not cleaved from the array.

In some embodiments, a capture probe can be extended (an “extended capture probe,” e.g., as described herein). For example, extending a capture probe can include generating cDNA from a captured (hybridized) RNA. This process involves synthesis of a complementary strand of the hybridized nucleic acid, e.g., generating cDNA based on the captured RNA template (the RNA hybridized to the capture domain of the capture probe). Thus, in an initial step of extending a capture probe, e.g., the cDNA generation, the captured (hybridized) nucleic acid, e.g., RNA, acts as a template for the extension, e.g., reverse transcription, step.

In some embodiments, the capture probe is extended using reverse transcription. For example, reverse transcription includes synthesizing cDNA (complementary or copy DNA) from RNA, e.g., (messenger RNA), using a reverse transcriptase. In some embodiments, reverse transcription is performed while the tissue is still in place, generating an analyte library, where the analyte library includes the spatial barcodes from the adjacent capture probes. In some embodiments, the capture probe is extended using one or more DNA polymerases.

In some embodiments, a capture domain of a capture probe includes a primer for producing the complementary strand of a nucleic acid hybridized to the capture probe, e.g., a primer for DNA polymerase and/or reverse transcription. The nucleic acid, e.g., DNA and/or cDNA, molecules generated by the extension reaction incorporate the sequence of the capture probe. The extension of the capture probe, e.g., a DNA polymerase and/or reverse transcription reaction, can be performed using a variety of suitable enzymes and protocols.

In some embodiments, a full-length DNA (e.g., cDNA) molecule is generated. In some embodiments, a “full-length” DNA molecule refers to the whole of the captured nucleic acid molecule. However, if a nucleic acid (e.g., RNA) was partially degraded in the tissue sample, then the captured nucleic acid molecules will not be the same length as the initial RNA in the tissue sample. In some embodiments, the 3′ end of the extended probes, e.g., first strand cDNA molecules, is modified. For example, a linker or adaptor can be ligated to the 3′ end of the extended probes. This can be achieved using single stranded ligation enzymes such as T4 RNA ligase or Circligase™ (available from Lucigen, Middleton, WI). In some embodiments, template switching oligonucleotides are used to extend cDNA in order to generate a full-length cDNA (or as close to a full-length cDNA as possible). In some embodiments, a second strand synthesis helper probe (a partially double stranded DNA molecule capable of hybridizing to the 3′ end of the extended capture probe), can be ligated to the 3′ end of the extended probe, e.g., first strand cDNA, molecule using a double stranded ligation enzyme such as T4 DNA ligase. Other enzymes appropriate for the ligation step are known in the art and include, e.g., Tth DNA ligase, Taq DNA ligase, Thermococcus sp. (strain 9° N) DNA ligase (9° N™ DNA ligase, New England Biolabs), Ampligase™ (available from Lucigen, Middleton, WI), and SplintR (available from New England Biolabs, Ipswich, MA). In some embodiments, a polynucleotide tail, e.g., a poly(A) tail, is incorporated at the 3′ end of the extended probe molecules. In some embodiments, the polynucleotide tail is incorporated using a terminal transferase active enzyme.

In some embodiments, double-stranded extended capture probes are treated to remove any unextended capture probes prior to amplification and/or analysis, e.g., sequence analysis. This can be achieved by a variety of methods, e.g., using an enzyme to degrade the unextended probes, such as an exonuclease enzyme, or purification columns.

In some embodiments, extended capture probes are amplified to yield quantities that are sufficient for analysis, e.g., via DNA sequencing. In some embodiments, the first strand of the extended capture probes (e.g., DNA and/or cDNA molecules) acts as a template for the amplification reaction (e.g., a polymerase chain reaction).

In some embodiments, the amplification reaction incorporates an affinity group onto the extended capture probe (e.g., RNA-cDNA hybrid) using a primer including the affinity group. In some embodiments, the primer includes an affinity group and the extended capture probes includes the affinity group. The affinity group can correspond to any of the affinity groups described previously.

In some embodiments, the extended capture probes including the affinity group can be coupled to a substrate specific for the affinity group. In some embodiments, the substrate can include an antibody or antibody fragment. In some embodiments, the substrate includes avidin or streptavidin and the affinity group includes biotin. In some embodiments, the substrate includes maltose and the affinity group includes maltose-binding protein. In some embodiments, the substrate includes maltose-binding protein and the affinity group includes maltose. In some embodiments, amplifying the extended capture probes can function to release the extended probes from the surface of the substrate, insofar as copies of the extended probes are not immobilized on the substrate.

In some embodiments, the extended capture probe or complement or amplicon thereof is released. The step of releasing the extended capture probe or complement or amplicon thereof from the surface of the substrate can be achieved in a number of ways. In some embodiments, an extended capture probe or a complement thereof is released from the array by nucleic acid cleavage and/or by denaturation (e.g., by heating to denature a double-stranded molecule).

In some embodiments, the extended capture probe or complement or amplicon thereof is released from the surface of the substrate (e.g., array) by physical means. For example, where the extended capture probe is indirectly immobilized on the array substrate, e.g., via hybridization to a surface probe, it can be sufficient to disrupt the interaction between the extended capture probe and the surface probe. Methods for disrupting the interaction between nucleic acid molecules include denaturing double stranded nucleic acid molecules are known in the art. A straightforward method for releasing the DNA molecules (e.g., or i.e., of stripping the array of extended probes) is to use a solution that interferes with the hydrogen bonds of the double stranded molecules. In some embodiments, the extended capture probe is released by an applying heated solution, such as water or buffer, of at least 85° C., e.g., at least 90, 91, 92, 93, 94, 95, 96, 97, 98, or 99° C. In some embodiments, a solution including salts, surfactants, etc. that can further destabilize the interaction between the nucleic acid molecules is added to release the extended capture probe from the substrate.

In some embodiments, where the extended capture probe includes a cleavage domain, the extended capture probe is released from the surface of the substrate by cleavage. For example, the cleavage domain of the extended capture probe can be cleaved by any of the methods described herein. In some embodiments, the extended capture probe is released from the surface of the substrate, e.g., via cleavage of a cleavage domain in the extended capture probe, prior to the step of amplifying the extended capture probe.

In some embodiments, probes complementary to the extended capture probe can be contacted with the substrate. In some embodiments, the biological sample can be in contact with the substrate when the probes are contacted with the substrate. In some embodiments, the biological sample can be removed from the substrate prior to contacting the substrate with probes. In some embodiments, the probes can be labeled with a detectable label (e.g., any of the detectable labels described herein). In some embodiments, probes that do not specially bind (e.g., hybridize) to an extended capture probe can be washed away. In some embodiments, probes complementary to the extended capture probe can be detected on the substrate (e.g., imaging, any of the detection methods described herein).

In some embodiments, probes complementary to an extended capture probe can be about 4 nucleotides to about 100 nucleotides long. In some embodiments, probes (e.g., detectable probes) complementary to an extended capture probe can be about 10 nucleotides to about 90 nucleotides long. In some embodiments, probes (e.g., detectable probes) complementary to an extended capture probe can be about 20 nucleotides to about 80 nucleotides long. In some embodiments, probes (e.g., detectable probes) complementary to an extended capture probe can be about 30 nucleotides to about 60 nucleotides long. In some embodiments, probes (e.g., detectable probes) complementary to an extended capture probe can be about 40 nucleotides to about 50 nucleotides long. In some embodiments, probes (e.g., detectable probes) complementary to an extended capture probe can be about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 nucleotides long.

In some embodiments, about 1 to about 100 probes can be contacted to the substrate and specifically bind (e.g., hybridize) to an extended capture probe. In some embodiments, about 1 to about 10 probes can be contacted to the substrate and specifically bind (e.g., hybridize) to an extended capture probe. In some embodiments, about 10 to about 100 probes can be contacted to the substrate and specifically bind (e.g., hybridize) to an extended capture probe. In some embodiments, about 20 to about 90 probes can be contacted to the substrate and specifically bind (e.g., hybridize) to an extended capture probe. In some embodiments, about 30 to about 80 probes (e.g., detectable probes) can be contacted to the substrate and specifically bind (e.g., hybridize) to an extended capture probe. In some embodiments, about 40 to about 70 probes can be contacted to the substrate and specifically bind (e.g., hybridize) to an extended capture probe. In some embodiments, about 50 to about 60 probes can be contacted to the substrate and specifically bind (e.g., hybridize) to an extended capture probe. In some embodiments, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 51, about 52, about 53, about 54, about 55, about 56, about 57, about 58, about 59, about 60, about 61, about 62, about 63, about 64, about 65, about 66, about 67, about 68, about 69, about 70, about 71, about 72, about 73, about 74, about 75, about 76, about 77, about 78, about 79, about 80, about 81, about 82, about 83, about 84, about 85, about 86, about 87, about 88, about 89, about 90, about 91, about 92, about 93, about 94, about 95, about 96, about 97, about 98, and about 99 probes can be contacted to the substrate and specifically bind (e.g., hybridize) to an extended capture probe.

In some embodiments, the probes can be complementary to a single analyte (e.g., a single gene). In some embodiments, the probes can be complementary to one or more analytes (e.g., analytes in a family of genes). In some embodiments, the probes (e.g., detectable probes) can be for a panel of genes associated with a disease (e.g., cancer, Alzheimer's disease, Parkinson's disease).

In some instances, the analyte and capture probe can be amplified or copied, creating a plurality of cDNA molecules. In some embodiments, cDNA can be denatured from the capture probe template and transferred (e.g., to a clean tube) for amplification, and/or library construction. The spatially-barcoded cDNA can be amplified via PCR prior to library construction. The cDNA can then be enzymatically fragmented and size-selected in order to optimize for cDNA amplicon size. P5 and P7 sequences directed to capturing the amplicons on a sequencing flowcell (Illumina sequencing instruments) can be appended to the amplicons, i7, and i5 can be used as sample indexes, and TruSeq Read 2 can be added via End Repair, A-tailing, Adaptor Ligation, and PCR. The cDNA fragments can then be sequenced using paired-end sequencing using TruSeq Read 1 and TruSeq Read 2 as sequencing primer sites. The additional sequences are directed toward Illumina sequencing instruments or sequencing instruments that utilize those sequences; however a skilled artisan will understand that additional or alternative sequences used by other sequencing instruments or technologies are also equally applicable for use in the aforementioned methods.

In some embodiments, where a sample is barcoded directly via hybridization with capture probes or analyte capture agents hybridized, bound, or associated with either the cell surface, or introduced into the cell, as described above, sequencing can be performed on the intact sample.

A wide variety of different sequencing methods can be used to analyze a barcoded analyte or derivative thereof. In general, sequenced polynucleotides can be, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog).

Sequencing of polynucleotides can be performed by various systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based single plex methods, emulsion PCR), and/or isothermal amplification. Non-limiting examples of methods for sequencing genetic material include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods.

### (v) Analyte Analysis on the First Substrate and Second Substrate Using Templated Ligation

In some instances, instead of capturing the analytes, a derivative of an analyte is captured. In some instances, the derivative of the analyte is a ligation product that comprises two or more nucleic acid sequences that hybridize to adjacent sequences of the analyte. Once templated ligation is performed, the biological sample can be permeabilized and both substrates can capture the ligation product (similar to capture of an analyte). Capture of the ligation product is similar to capture of the analyte because the ligation product comprises a poly(A) tail similar to an mRNA analyte. Additional features of templated ligation are now provided.

Templated ligation or RNA-templated ligation (RTL) is a process that includes multiple oligonucleotides (also called “oligonucleotide probes” or simply “probes,” and a pair of probes can be called interchangeably “first probes” and “second probes,” or “first probe oligonucleotides” and “second probe oligonucleotides,”) that hybridize to adjacent complementary analyte (e.g., mRNA) sequences. Upon hybridization, the two oligonucleotides are ligated to one another, creating a ligation product in the event that both oligonucleotides hybridize to their respective complementary sequences. In some instances, at least one of the oligonucleotides includes a sequence (e.g., a poly-adenylation sequence) that can be hybridized to a probe on an array described herein (e.g., the probe comprises a poly-thymine sequence in some instances). In some instances, prior to hybridization of the poly-thymine to the poly(A) sequence, an endonuclease digests the analyte that is hybridized to the ligation product. This step frees the newly formed ligation product to hybridize to a capture probe on a spatial array. In this way, templated ligation provides a method to perform targeted RNA capture on a spatial array.

Targeted RNA capture allows for examination of a subset of RNA analytes from the entire transcriptome. In some embodiments, the subset of analytes includes an individual target RNA. In some instances, the presence of the ligation product that is created as a result of the templated ligation methods described herein indicates that the individual target RNA is present. In some instances, the absence of the ligation product that is created as a result of the templated ligation methods described herein indicates that the individual target RNA is not present. In some instances, an absence of the ligation product is because one of the oligonucleotide probes did not hybridize to the analyte. In some instances, an absence of the ligation product is because both (e.g., two) of the oligonucleotide probes did not hybridize to the analyte.

In some embodiments, the subset of analytes detected using methods disclosed herein includes two or more (e.g., 2, 3, 4, 5, 6, 7, 8, 9, 10, or more) targeted RNAs. In some embodiments, the subset of analytes includes one or more mRNAs transcribed by a single gene. In some embodiments, the subset of analytes includes one or more mRNAs transcribed by more than one targeted genes. In some embodiments, the subset of analytes includes one or more mRNA splice variants of one or more targeted genes. In some embodiments, the subset of analytes includes non-polyadenylated RNAs in a biological sample. In some embodiments, the subset of analytes includes detection of mRNAs having one or more (e.g., 2, 3, 4, 5, or more) single nucleotide polymorphisms (SNPs) in a biological sample.

In some embodiments, the subset of analytes includes mRNAs that mediate expression of a set of genes of interest. For example, in some instances, the subset of analytes detected using the templated ligation methods disclosed herein include analytes that are translated into transcription factors that control one or more cellular pathways. In some embodiments, the subset of analytes includes mRNAs that share identical or substantially similar sequences, which mRNAs are translated into polypeptides having similar functional groups or protein domains. In some embodiments, the subset of analytes includes mRNAs that do not share identical or substantially similar sequences, which mRNAs are translated into proteins that do not share similar functional groups or protein domains. In some embodiments, the subset of analytes includes mRNAs that are translated into proteins that function in the same or similar biological pathways. In some embodiments, the biological pathways are associated with a pathologic disease. For example, targeted RNA capture can detect genes that are overexpressed or underexpressed in a cancer sample.

In some embodiments, the subset of analytes includes 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, about 55, about 60, about 65, about 70, about 75, about 80, about 85, about 90, about 95, about 100, about 110, about 120, about 130, about 140, about 150, about 160, about 170, about 180, about 190, about 200, about 225, about 250, about 275, about 300, about 325, about 350, about 375, about 400, about 425, about 450, about 475, about 500, about 600, about 700, about 800, about 900, about 1000, or more analytes.

In some embodiments, the subset of analytes detected by targeted RNA capture methods provided herein includes a large proportion of the transcriptome of one or more cells. For example, the subset of analytes detected by targeted RNA capture methods provided herein can include at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or more of the mRNAs present in the transcriptome of one or more cells.

Methods disclosed herein can be performed on any type of sample. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some embodiments, a biological sample (e.g., tissue section) can be fixed with methanol, stained with hematoxylin and eosin, and imaged. In some embodiments, fixing, staining, and imaging occurs before one or more oligonucleotide probes are hybridized to the sample. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the slide.

In some embodiments, the methods of targeted RNA capture as disclosed herein include hybridization of multiple probe oligonucleotides. In some embodiments, the methods include 2, 3, 4, or more probe oligonucleotides that hybridize to one or more analytes of interest. In some embodiments, the methods include two probe oligonucleotides. In some embodiments, the probe oligonucleotide includes sequences complementary that are complementary or substantially complementary to an analyte. For example, in some embodiments, the probe oligonucleotide includes a sequence that is complementary or substantially complementary to an analyte (e.g., an mRNA of interest (e.g., to a portion of the sequence of an mRNA of interest)). Methods provided herein may be applied to a single nucleic acid molecule or a plurality of nucleic acid molecules. A method of analyzing a sample comprising a nucleic acid molecule may comprise providing a plurality of nucleic acid molecules (e.g., RNA molecules), where each nucleic acid molecule comprises a first target region (e.g., a sequence that is 3′ of a target sequence or a sequence that is 5′ of a target sequence) and a second target region (e.g., a sequence that is 5′ of a target sequence or a sequence that is 3′ of a target sequence), a plurality of first probe oligonucleotides, and a plurality of second probe oligonucleotides.

In some embodiments, the templated ligation methods that allow for targeted RNA capture as provided herein include a first probe oligonucleotide and a second probe oligonucleotide. The first and second probe oligonucleotides each include sequences that are substantially complementary to the sequence of an analyte of interest. By substantially complementary, it is meant that the first and/or second probe oligonucleotide is at least 70%, at least 75%, at least 80%, at least 85%, at least 90%, at least 91%, at least 92%, at least 93%, at least 94%, at least 95%, at least 96%, at least 97%, at least 98%, at least 99%, or 100% complementary to a sequence in an analyte. In some instances, the first probe oligonucleotide and the second probe oligonucleotide hybridize to adjacent sequences on an analyte.

In some embodiments, the first and/or second probe as disclosed herein includes one of at least two ribonucleic acid bases at the 3′ end; a functional sequence; a phosphorylated nucleotide at the 5′ end; and/or a capture probe binding domain. In some embodiments, the functional sequence is a primer sequence. The capture probe binding domain is a sequence that is complementary to a particular capture domain present in a capture probe. In some embodiments, the capture probe binding domain includes a poly(A) sequence. In some embodiments, the capture probe binding domain includes a poly-uridine sequence, a poly-thymidine sequence, or both. In some embodiments, the capture probe binding domain includes a random sequence (e.g., a random hexamer or octamer). In some embodiments, the capture probe binding domain is complementary to a capture domain in a capture probe that detects a particular target(s) of interest.

In some embodiments, a capture probe binding domain blocking moiety that interacts with the capture probe binding domain is provided. In some instances, the capture probe binding domain blocking moiety includes a nucleic acid sequence. In some instances, the capture probe binding domain blocking moiety is a DNA oligonucleotide. In some instances, the capture probe binding domain blocking moiety is an RNA oligonucleotide. In some embodiments, a capture probe binding domain blocking moiety includes a sequence that is complementary or substantially complementary to a capture probe binding domain. In some embodiments, a capture probe binding domain blocking moiety prevents the capture probe binding domain from binding the capture probe when present. In some embodiments, a capture probe binding domain blocking moiety is removed prior to binding the capture probe binding domain (e.g., present in a ligated probe) to a capture probe. In some embodiments, a capture probe binding domain blocking moiety comprises a poly-uridine sequence, a poly-thymidine sequence, or both.

In some embodiments, the first probe oligonucleotide hybridizes to an analyte. In some embodiments, the second probe oligonucleotide hybridizes to an analyte. In some embodiments, both the first probe oligonucleotide and the second probe oligonucleotide hybridize to an analyte. Hybridization can occur at a target having a sequence that is 100% complementary to the probe oligonucleotide(s). In some embodiments, hybridization can occur at a target having a sequence that is at least (e.g., at least about) 80%, at least (e.g. at least about) 85%, at least (e.g. at least about) 90%, at least (e.g. at least about) 95%, at least (e.g. at least about) 96%, at least (e.g. at least about) 97%, at least (e.g. at least about) 98%, or at least (e.g. at least about) 99% complementary to the probe oligonucleotide(s).

After hybridization of the first and second probe oligonucleotides, in some embodiments, the first probe oligonucleotide is extended. After hybridization, in some embodiments, the second probe oligonucleotide is extended. Extending probes can be accomplished using any method disclosed herein. In some instances, a polymerase (e.g., a DNA polymerase) extends the first and/or second oligonucleotide.

In some embodiments, methods disclosed herein include a wash step. In some instances, the wash step occurs after hybridizing the first and the second probe oligonucleotides. The wash step removes any unbound oligonucleotides and can be performed using any technique or solution disclosed herein or known in the art. In some embodiments, multiple wash steps are performed to remove unbound oligonucleotides.

In some embodiments, after hybridization of probe oligonucleotides (e.g., first and the second probe oligonucleotides) to the analyte, the probe oligonucleotides (e.g., the first probe oligonucleotide and the second probe oligonucleotide) are ligated together, creating a single ligated probe that is complementary to the analyte. Ligation can be performed enzymatically or chemically, as described herein.

### (c) Biological Samples and Analytes

Methods disclosed herein can be performed on any type of sample. In some embodiments, the sample is a solid tissue sample. In some embodiments, the sample is a fresh tissue. In some embodiments, the sample is a frozen sample. In some embodiments, the sample was previously frozen, e.g., is a fresh frozen sample. In some embodiments, the sample is a formalin-fixed, paraffin embedded (FFPE) sample. As used herein, the terms “sample” and “biological sample” are interchangeable.

A “biological sample” can be obtained from the subject for analysis using any of a variety of techniques including, but not limited to, biopsy, surgery, and laser capture microscopy (LCM), and generally includes cells and/or other biological material from the subject. In addition to the subjects described above, a biological sample can be obtained from non-mammalian organisms (e.g., a plants, an insect, an arachnid, a nematode (e.g., Caenorhabditis elegans), a fungi, an amphibian, or a fish (e.g., zebrafish)). A biological sample can be obtained from a prokaryote such as a bacterium, e.g., Escherichia coli, Staphylococci or Mycoplasma pneumoniae; an archaea; a virus such as Hepatitis C virus or human immunodeficiency virus; or a viroid. A biological sample can be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

Biological samples can be derived from a homogeneous culture or population of the subjects or organisms mentioned herein or alternatively from a collection of several different organisms, for example, in a community or ecosystem.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient (see, e.g., U.S. Patent Application Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hypersegmented neutrophils), monocytes/macrophages, mast cells, thrombocytes/megakaryocytes, and dendritic cells.

The biological sample can include any number of macromolecules, for example, cellular macromolecules and organelles (e.g., mitochondria and nuclei). The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a check swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions.

Cell-free biological samples can include extracellular polynucleotides. Extracellular polynucleotides can be isolated from a bodily sample, e.g., blood, plasma, serum, urine, saliva, mucosal excretions, sputum, stool, and tears.

As discussed above, a biological sample can include a single analyte of interest, or more than one analyte of interest. Methods for performing multiplexed assays to analyze two or more different analytes in a single biological sample is discussed in a subsequent section of this disclosure.

Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. A diseased cell can have altered metabolic properties, gene expression, protein expression, and/or morphologic features. Examples of diseases include inflammatory disorders, metabolic disorders, nervous system disorders, and cancer. In some instances, the biological sample includes cancer or tumor cells. Cancer cells can be derived from solid tumors, hematological malignancies, cell lines, or obtained as circulating tumor cells. In some instances, the biological sample is a heterogenous sample. In some instances, the biological sample is a heterogenous sample that includes tumor or cancer cells and/or stromal cells,

In some instances, the cancer is breast cancer. In some instances, the breast cancer is triple positive breast cancer (TPBC). In some instances, the breast cancer is triple negative breast cancer (TNBC).

In some instances, the cancer is colorectal cancer. In some instances, the cancer is ovarian cancer. In certain embodiments, the cancer is squamous cell cancer, small-cell lung cancer, non-small cell lung cancer, gastrointestinal cancer, Hodgkin's or non-Hodgkin's lymphoma, pancreatic cancer, glioblastoma, glioma, cervical cancer, ovarian cancer, liver cancer, bladder cancer, breast cancer, colon cancer, colorectal cancer, endometrial carcinoma, myeloma, salivary gland carcinoma, kidney cancer, basal cell carcinoma, melanoma, prostate cancer, vulval cancer, thyroid cancer, testicular cancer, esophageal cancer, or a type of head or neck cancer. In certain embodiments, the cancer treated is desmoplastic melanoma, inflammatory breast cancer, thymoma, rectal cancer, anal cancer, or surgically treatable or non-surgically treatable brain stem glioma. In some embodiments, the subject is a human.

FFPE samples generally are heavily cross-linked and fragmented, and therefore this type of sample allows for limited RNA recovery using conventional detection techniques. In certain embodiments, methods of targeted RNA capture provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach.

In some instances, FFPE samples are stained (e.g., using H&E). The methods disclosed herein are compatible with H&E will allow for morphological context overlaid with transcriptomic analysis. However, depending on the need some samples may be stained with only a nuclear stain, such as staining a sample with only hematoxylin and not eosin, when location of a cell nucleus is needed.

In some embodiments, a biological sample (e.g. tissue section) can be fixed with methanol, stained with hematoxylin and eosin, and imaged. In some embodiments, fixing, staining, and imaging occurs before one or more analytes are captured. Some embodiments of any of the workflows described herein can further include a destaining step (e.g., a hematoxylin and eosin destaining step), after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer including HCl). The images can be used to map spatial gene expression patterns back to the biological sample. A permeabilization enzyme can be used to permeabilize the biological sample directly on the substrate.

In some embodiments, the FFPE sample is deparaffinized, permeabilized, equilibrated, and blocked before analyte capture. In some embodiments, deparaffinization using xylenes. In some embodiments, deparaffinization includes multiple washes with xylenes. In some embodiments, deparaffinization includes multiple washes with xylenes followed by removal of xylenes using multiple rounds of graded alcohol followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, permeabilizing a sample includes washing the sample with a phosphate buffer. In some embodiments, the buffer is PBS. In some embodiments, the buffer is PBST.

In some instances, the methods disclosed herein include preparation of a biological sample. In some instances, the biological sample is a tissue sample. In some instances, the biological sample is a tissue section. In some instances, the tissue is a fresh sample. In some instances, the fresh sample has been sectioned. In some instances, the tissue is a frozen sample. In some instances, the frozen sample has been sectioned. In some instances, the tissue is a fixed sample. In some instances, the tissue is a formalin-fixed paraffin-embedded (FFPE) tissue, a PFA fixed sample or an acetone fixed sample. Any other suitable tissue samples described herein can also be used in the methods.

In some instances, the biological sample is a live sample. In some instances, the biological sample is a section of a live tissue sample. In some instances, the biological sample is a culture of cells (e.g., as disclosed herein). Live sample as used herein refers to a sample that maintains at least 60%, 65%, 70%, 75%, 80%, 85%, 90%, 91%, 92%, 93%, 94%, 95%, 96%, 97%, 98%, 99% or 100% viability. In some instances, the live tissue is sectioned using a vibratome.

The apparatus, systems, methods, and compositions described in this disclosure can be used to detect and analyze a wide variety of different analytes. For the purpose of this disclosure, an “analyte” can include any biological substance, structure, moiety, or component to be analyzed. The term “target” can similarly refer to an analyte of interest. In some instances, the different analytes (e.g., the first analyte and the second analyte) can be protein and nucleic acid (e.g., RNA or DNA). Additionally, or in the alternative, the different analytes can be intermediate agent or portion thereof and RNA. The intermediate agent can be a connected probe (e.g., RTL probe) or analyte capture agent (e.g., oligo-conjugated antibody). Additionally, or in the alternative, the different analytes can be RNA and DNA. Additionally, or in the alternative, the different analytes can be intermediate agent and DNA. Additionally, or in the alternative, the different analytes can be targeted capture on one slide and whole transcriptome or DNA on other slide.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte can be an organelle (e.g., nuclei or mitochondria).

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V (D) J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). In some embodiments, a perturbation agent can be a small molecule, an antibody, a drug, an aptamer, a miRNA, a physical environmental (e.g., temperature change), or any other known perturbation agents.

In certain embodiments, an analyte can be extracted from a live cell. Processing conditions can be adjusted to ensure that a biological sample remains live during analysis, and analytes are extracted from (or released from) live cells of the sample. Live cell-derived analytes can be obtained only once from the sample, or can be obtained at intervals from a sample that continues to remain in viable condition.

In general, the systems, apparatus, methods, and compositions can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

As used herein, the analytes captured using the two-substrate gene expression array system can be analytes in the same location and having the same or similar sequence. In some instances, the analytes captured at a particular spot are different (e.g., they are different genes or have different and/or non-conserved sequences). It is appreciated that because the number of analytes detected is at least doubled because two gene expression assays are used, then both similar and non-similar (e.g., non-conserved) analytes can be detected.

### (d) Kits, Systems, and Compositions

In some embodiments, also provided herein are kits, systems, and apparatuses, each configured to perform the methods disclosed herein.

In some instances, the kits and systems include one or more reagents to detect one or more analytes described herein. In some instances, the kits and systems include a first substrate comprising a plurality of capture probes, each of which includes a capture domain (e.g., a poly(T) sequence. In some instances, the kits and systems include a second substrate comprising a plurality of capture probes, each of which includes a capture domain (e.g., a poly(T) sequence and a spatial barcode. It is appreciated that any of the capture probes disclosed herein can be designed so that a user can detect any analyte of interest.

A non-limiting example of a kit used to perform any of the methods described herein includes: (a) a first substrate comprising a plurality of first capture probes, wherein a first capture probe of the plurality of first capture probes comprises a first capture domain and a first spatial barcode; (b) a second substrate comprising a plurality of second capture probes, wherein a second capture probe of the plurality of second capture probes comprises (i) a spatial barcode and (ii) a second capture domain; and (c) instructions for performing any of the methods disclosed herein.

A non-limiting example of a kit used to perform any of the methods described herein includes: (a) a first substrate comprising a plurality of first capture probes, wherein a first capture probe of the plurality of first capture probes comprises a first capture domain and a first spatial barcode; (b) a second substrate comprising a plurality of second capture probes, wherein a second capture probe of the plurality of second capture probes comprises (i) a spatial barcode and (ii) a second capture domain; and (c) the biological sample.

It is further appreciated that the kits, systems, and apparatuses can include one or more reagents necessary to perform any of the methods disclosed herein. In addition, the kits, systems, and apparatuses can also include one or more enzymes (e.g., polymerase; reverse transcriptase; ligase) necessary to perform any of the methods disclosed herein.

### Examples

### EXAMPLE 1-Detection of Multiple Analytes in a Biological Sample Using Two Gene Expression Arrays

This example provides an exemplary method for detection of multiple analytes in a biological sample using two gene expression arrays.

a. Experimental and Control Setup

Two control set ups were utilized. In the first control sample, as shown in FIG. 7A (top left), a biological sample 704 was placed on directly on a spatially barcoded substrate (e.g., gene expression substrate) 702. Analytes 706 were captured as described below. In the second control sample, as shown in FIG. 7A (top right), a biological sample 710 is placed on a tissue substrate (e.g., a glass tissue slide) 708. The tissue substrate 708 then was sandwiched with a spatially barcoded substrate (e.g., gene expression substrate) 712. After sandwiching, analytes 714 from the biological sample 710 migrated 716 to capture probes 718 on the spatially barcoded substrate (e.g., gene expression substrate) 712. The capture probes 718 comprise (i) a capture domain (e.g., or i.e., a poly-thymine sequence) that hybridizes to the poly(A) tail of an analyte 714 and (ii) a spatial barcode.

Two test conditions—each using two gene expression substrates-were run. In one test condition, as shown in FIG. 7A (bottom left), the biological sample 724 was placed on the top gene expression substrate 720, which comprises a lawn of capture probes 722, each comprising a capture domain and a spatial barcode. The top gene expression substrate 720 then was sandwiched with a second, bottom gene expression substrate 730, which comprised its own lawn of capture probes 732, each comprising a capture domain and a spatial barcode. Analytes 726 from the biological sample 724 migrated 728 from the top gene expression substrate 720 to the bottom gene expression substrate 730. At the same time, analytes 724 were captured on the top gene expression substrate 720 by capture probes 722.

In a second test condition, as shown in FIG. 7A (bottom right), the biological sample 742 was placed on the bottom gene expression substrate 738, which comprises a lawn of capture probes 740, each comprising a capture domain and a spatial barcode. The bottom gene expression substrate 738 then was sandwiched with a second, top gene expression substrate 734, which comprised its own lawn of capture probes 736, each comprising a capture domain and a spatial barcode. Analytes 744 from the biological sample 742 migrated 746 from the bottom gene expression substrate 738 to the top gene expression substrate 734. At the same time, analytes 744 were captured on the bottom gene expression substrate 738 by capture probes 740.

As an alternative view, as shown in FIG. 7B, a biological sample 752 was placed on a first gene expression substrate 750. A second gene expression substrate 756 was sandwiched 754 with the first gene expression substrate 750. Analytes from the biological sample 752 were captured by capture probes 758 and 760 on each gene expression substrate. After, the gene expression substrates were separated, and methods of spatial analysis were performed to identify the location and abundance of analytes in the biological sample 752.

b. Detection of Multiple Analytes in a Fresh Frozen Biological Sample using Two Gene Expression Arrays

Using the control and experimental setups described in Example 1 (a) above, a 10 μm fresh frozen mouse brain sample was sectioned and placed on one of the substrates four substrates (two control substrates and two test substrates).

In the sandwich assays, after placing the biological sample onto the first substrate (either top or bottom). Briefly, a 10 μm tissue sample was sectioned and placed on a first substrate. After placing the biological sample on the first substrate, a second substrate is placed either (A) on top, superior to, the first substrate, or (B) below, or inferior to, the first substrate, creating a sandwich configuration with the biological sample between the first substrate and the second substrate. Each substrate in the sandwich has a plurality of capture probes, each of which includes both a capture domain (e.g., a poly-thymine sequence) and a spatial domain. A permeabilization buffer comprising proteinase K was added to the biological sample, allowing the analytes to migrate from the biological sample.

Analytes migrated passively towards both substrates and were captured by capture probes each of the two substrates. Analytes captured on each substrate were prepared for analysis. In brief, the captured analytes on the first and second substrate were immobilized on each substrate via the poly(A) tail of the analyte (e.g., or i.e., the poly(A) tail hybridized to the poly-thymine sequence of the capture probe). After hybridization, the sandwich setup was broken. That is, each substrate was separated and further experiments were performed. In particular, after separation, the captured analytes were copied, using the analyte as a template; and the extension product was released from the spatial array. Briefly, the tissues were incubated with a second strand extension mix comprising DNA polymerase for 25 minutes at 53° C. Following incubation, the second strand extension mix was removed from the tissues and the tissues were washed with 2×SSC. A solution of KOH was added to the tissue at room temperature for 10 minutes to release the extension product from each spatial array and the supernatant from each tissue well was transferred for quantification, and library preparation. Sample quantification was performed using qPCR and KAPA SYBR FAST qPCR master mix according to the manufacturer's instructions. For library preparation, samples was indexed using an Amp Mix that included dual indexing primers and an Amp Mix. Nucleic acids from the amplification reaction were then sequenced and analyzed. An image of the sample was generated based on analyte detection.

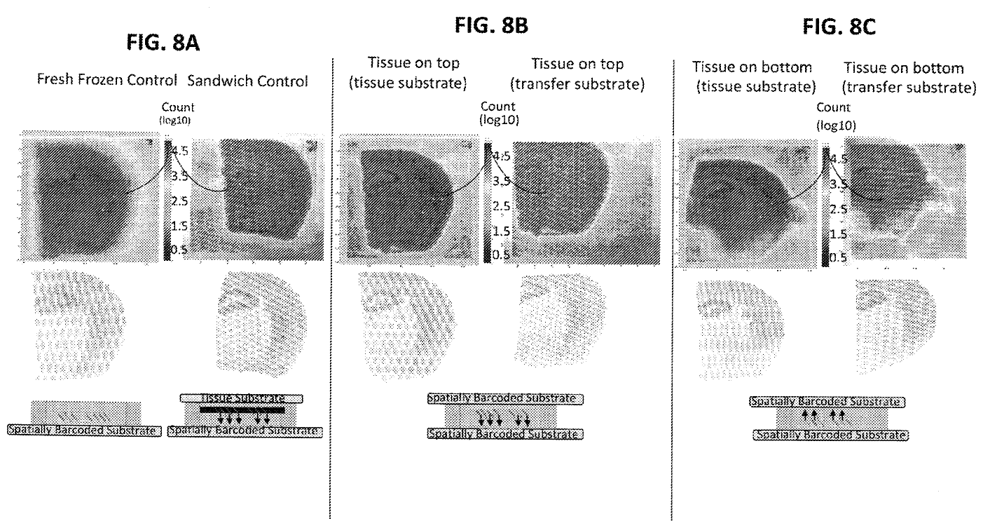

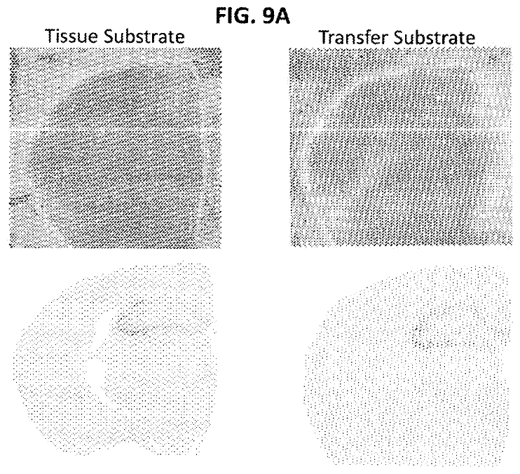

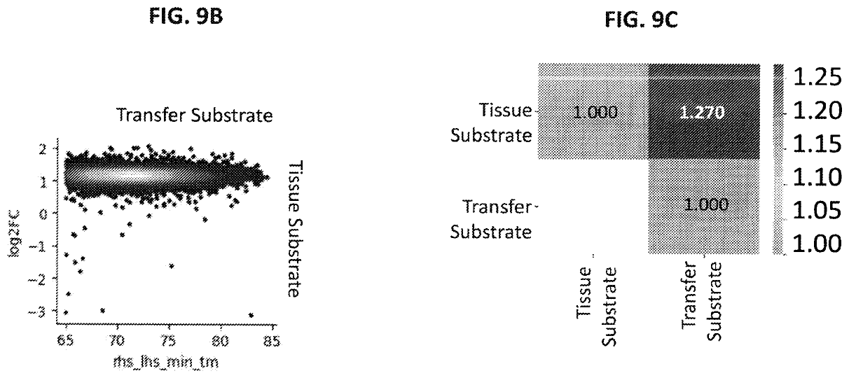

As shown in FIGS. 8A-8C, analytes were detected in each of the controls (FIG. 8A) and each of the test samples (FIGS. 8B and 8C). In particular, in the test condition, analytes were readily detected in both the tissue substrate (e.g., or i.e., the first gene expression substrate) and the transfer gene expression substrate (e.g., or i.e., the second gene expression substrate). FIG. 9A. Further, reads were compared between the tissue substrate (e.g., or i.e., the first gene expression substrate) and the transfer gene expression substrate (e.g., or i.e., the second gene expression substrate). As shown in FIGS. 9B and 9C, capture efficiency was relatively the same in both the tissue substrate (e.g., or i.e., the first gene expression substrate) and the transfer gene expression substrate (e.g., or i.e., the second gene expression substrate). These data are consistent with similar valid barcodes, valid UMIs, percent reads mapped confidently to the transcriptome, percent of fraction reads that were unmapped, percent of reads with full poly(A) sequence, the percent of fraction reads with any switch oligo sequence, the median genes per spot, the median UMI counts per spot, and the sequencing saturation, both between the two substrates in the sandwich test conditions and compared with the fresh frozen (non-sandwich control) and the sandwich control. See Tables 1 and 2. Taken together, these data demonstrate that a fresh frozen sample can be used is a sandwich assay comprising two gene expression substrates. Thus, the experiments illustrate that multiple analytes can be examined at the same position in a fresh frozen biological sample using two gene expression substrates.

TABLE 1
Analysis of Spatial Expression in Fresh Frozen Sample.

| | | | | | | Reads | Fraction reads |
|---|---|---|---|---|---|---|---|
| | | | | Reads mapped | Fraction | with full | with any |
| | | Valid | Valid | confidently to | reads | poly(A) | switch oligo |
| Experimental Setup | Substrate Analyzed | barcodes | UMIs | transcriptome | unmapped | sequence | sequence |
| Fresh Frozen Control | Single Substrate | 95.20% | 100.00% | 79.80% | 6.20% | 2.10% | 11.30% |
| Sandwich Control | Transfer Substrate | 95.20% | 100.00% | 79.10% | 7.10% | 3.10% | 10.40% |
| GEX to GEX - Tissue on Top Substrate | Tissue Substrate | 96.00% | 100.00% | 80.20% | 8.60% | 2.60% | 10.20% |
| GEX to GEX - Tissue on Top Substrate | Transfer Substrate | 96.10% | 100.00% | 78.10% | 10.00% | 3.80% | 11.90% |
| GEX to GEX - Tissue on Bottom Substrate | Tissue Substrate | 95.80% | 100.00% | 80.00% | 6.50% | 1.50% | 13.30% |
| GEX to GEX - Tissue on Bottom Substrate | Transfer Substrate | 94.60% | 100.00% | 75.10% | 9.20% | 2.30% | 12.60% |

TABLE 2
Analysis of Spatial Expression in Fresh Frozen Sample.

| | | mm10 Median | mm10 Median UMI | |
|---|---|---|---|---|
| | | genes per spot | counts per spot | |
| | | (30k raw reads per | (30k raw reads per | Sequencing |
| Experimental Setup | Substrate Analyzed | spot) | spot) | Saturation |
| Fresh Frozen Control | Single Substrate | 4362 | 12151 | 56.10% |
| Sandwich Control | Transfer Substrate | 4488 | 14041 | 48.20% |
| GEX to GEX - Tissue | Tissue Substrate | 4934 | 16599 | 42.10% |
| on Top Substrate | | | | |
| GEX to GEX - Tissue | Transfer Substrate | 4762 | 15685 | 44.70% |
| on Top Substrate | | | | |
| GEX to GEX - Tissue | Tissue Substrate | 4801 | 14832 | 47.00% |
| on Bottom Substrate | | | | |
| GEX to GEX - Tissue | Transfer Substrate | 4103 | 12930 | 56.20% |
| on Bottom Substrate | | | | |

c. Detection of Multiple Analytes in a FFPE Biological Sample using Two Gene Expression Arrays

Using the control and experimental setups described in Example 1 (a) above (control setups contained only the spatially barcoded polyT oligonucleotide while the test setups contained two capture sequences: spatially barcoded polyT oligonucleotides, and spatially barcoded oligonucleotides containing capture sequence 1, which is configured to hybridize to oligonucleotides of oligonucleotide-conjugated antibodies), a formalin fixed (FFPE) mouse brain was sectioned to a 10 μm thickness and placed upon their respective setup. The FFPE sectioned mouse brain tissue slides were then deparaffinized, decrosslinked, and equilibrated with a pre-Hybridization Buffer. Decrosslinking of the sample was performed using a TE decrosslinking reagent (TE buffer pH 9.0, 70° C.) (Genemed 10-0046). After decrosslinking, the samples were hybridized with gene-specific probes overnight, followed by washing steps, a ligation step, and further washing to remove ligated mismatches. The biological sample was then incubated with oligonucleotide-conjugated antibodies, followed by washes using PBST to remove excess antibodies. After, the biological sample was mounted onto the first substrate (either top or bottom). Once placing the biological sample on the first substrate, a second substrate was placed either (A) on top, superior to, the first substrate, or (B) below, or inferior to, the first substrate, creating a sandwich configuration with the biological sample between the first substrate and the second substrate. Each substrate in the sandwich has a plurality of capture probes, each of which includes both a capture domain (e.g., a poly-thymine sequence, capture sequence 1) and a spatial domain. A permeabilization buffer comprising proteinase K was added to the biological sample, allowing the analytes to migrate from the biological sample.

Analytes migrated passively towards both substrates and were captured by capture probes each of the two substrates. Analytes captured on each substrate were prepared for analysis. In brief, the captured analytes on the first and second substrate were immobilized on each substrate via the poly(A) tail of the analyte (e.g., or i.e., the poly(A) tail hybridized to the poly-thymine sequence of the capture probe). After hybridization, the sandwich setup was broken. That is, each substrate was separated and further experiments were performed. In particular, after separation, the captured analytes were copied, using the analyte as a template; and the extension product was released from the spatial array. Briefly, the tissues were incubated with a second strand extension mix comprising DNA polymerase for 25 minutes at 53° C. Following incubation, the second strand extension mix was removed from the tissues and the tissues were washed with 2×SSC. A solution of KOH was added to the tissue at room temperature for 10 minutes to release the extension product from each spatial array and the supernatant from each tissue well was transferred for quantification, and library preparation. Sample quantification was performed using qPCR and KAPA SYBR FAST qPCR master mix according to the manufacturer's instructions. For library preparation, samples was indexed using an Amp Mix that included dual indexing primers and an Amp Mix. Nucleic acids from the amplification reaction were then sequenced and analyzed. An image of the sample was generated based on analyte detection.

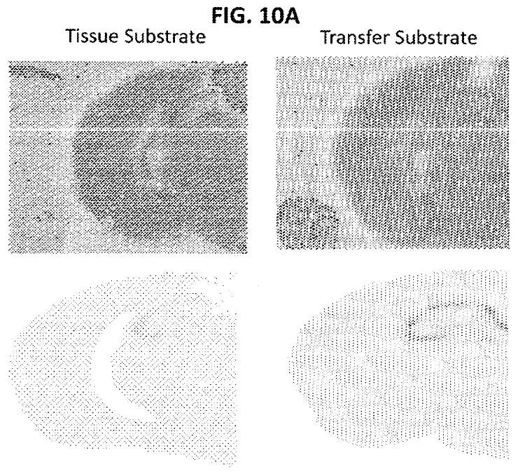

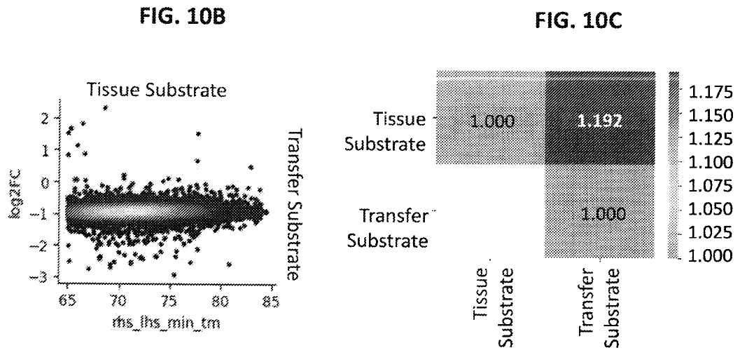

As shown in FIGS. 10A-10B, analytes were readily detected in both the tissue substrate (e.g., or i.e., the first gene expression substrate) and the transfer gene expression substrate (e.g., or i.e., the second gene expression substrate). Further, reads were compared between the tissue substrate (e.g., or i.e., the first gene expression substrate) and the transfer gene expression substrate (e.g., or i.e., the second gene expression substrate). As shown in FIGS. 10B and 10C, capture efficiency was relatively the same in both the tissue substrate (e.g., or i.e., the first gene expression substrate) and the transfer gene expression substrate (e.g., or i.e., the second gene expression substrate). These data are consistent with similar valid barcodes, valid UMIs, percent reads mapped confidently to the transcriptome, percent of fraction reads that were unmapped, percent of reads with full poly(A) sequence, the percent of fraction reads with any switch oligo sequence, the median genes per spot, the median UMI counts per spot, and the sequencing saturation, both between the two substrates in the sandwich test conditions and compared with the FFPE (non-sandwich control) and the sandwich control. See Tables 3 and 4. Furthermore, as noted in Table 3, antibody oligonucleotides were present in the setups shown. Taken together, these data demonstrate that an FFPE sample can be used is a sandwich assay comprising two gene expression substrates. Thus, the experiments illustrate that multiple analytes can be examined at the same position in a FFPE biological sample using two gene expression substrates.

TABLE 3
Analysis of Spatial Expression in FFPE Sample.

| | | | | Reads mapped | Fraction | | Fraction |
|---|---|---|---|---|---|---|---|
| | | Valid | Valid | confidently to | reads | Fraction of | targeted reads |
| Experimental Setup | Substrate Analyzed | Barcodes | UMIs | transcriptome | unmapped | chimeric reads | usable |
| FFPE Control | Single Substrate | 99.00% | 100% | 83.60% | 13.80% | 1.90% | 73.40% |
| Sandwich Control | Transfer Substrate | 99.00% | 100% | 96.40% | 1.80% | 1.40% | 79.50% |
| GEX to GEX - Tissue on Top Substrate | Tissue Substrate | 98.90% | 100% | 80.60% | 14.90% | 3.10% | 73.30% |
| GEX to GEX - Tissue on Top Substrate | Transfer Substrate | 99.00% | 100% | 96.10% | 1.70% | 1.70% | 82.90% |
| GEX to GEX - Tissue on Bottom Substrate | Tissue Substrate | 98.90% | 100% | 85.40% | 10.70% | 2.90% | 52.70% |
| GEX to GEX - Tissue on Bottom Substrate | Transfer Substrate | 99.00% | 100% | 96.40% | 1.70% | 1.40% | 83.80% |

*antibody oligonucleotides present in all setups shown

TABLE 4
Analysis of Spatial Expression in FFPE Sample.

| | | Fraction | | Median panel | Median panel | Panel cDNA | Panel cDNA |
|---|---|---|---|---|---|---|---|
| | | Reads in | Fraction | genes detected | UMI counts | PCR Duplication | PCR Duplication |
| | | Spots Under | targeted | at 5000 panel | at 5000 panel | (5000 panel | (5000 raw |
| Experimental Setup | Substrate Analyzed | Tissue | reads usable | reads per spot | reads per spot | reads per spot) | reads per spot) |
| FFPE Control | Single Substrate | 88.70% | 73.40% | 1674 | 2676 | 44.50% | 36.50% |
| Sandwich Control | Transfer Substrate | 83.30% | 79.50% | 1780 | 2945 | 36.10% | 30.20% |
| GEX to GEX - Tissue on | Tissue Substrate | 91.80% | 73.30% | 1339 | 2018 | 56.70% | 48.20% |
| Top Substrate | | | | | | | |
| GEX to GEX - Tissue on | Transfer Substrate | 87.20% | 82.90% | 1495 | 2352 | 49.30% | 44.20% |
| Top Substrate | | | | | | | |
| GEX to GEX - Tissue on | Tissue Substrate | 62.40% | 52.70% | 1500 | 2402 | 48.60% | 32.10% |
| Bottom Substrate | | | | | | | |
| GEX to GEX - Tissue on | Transfer Substrate | 87.90% | 83.80% | 1529 | 2504 | 45.70% | 41.10% |
| Bottom Substrate | | | | | | | |

### EXAMPLE 2: Efficient Analyte Capture from Slide-Mounted Fresh Frozen Mouse Brain Sections onto Spatial Array Slides

Analyte capture onto spatially barcoded arrays and subsequent sequencing was demonstrated under sandwich and non-sandwich conditions. For the test (sandwiching) condition, archived tissue-mounted standard glass slides containing hematoxylin/eosin stained fresh frozen mouse brain sections were used. For control (non-sandwich) condition, GEx array slides with hematoxylin/eosin stained fresh frozen mouse brain sections mounted directly onto the array area were used. Under both conditions, tissue sections were subjected to a hematoxylin destaining step. Slides processed according to the “sandwiching” condition were briefly dried at 37° C., then mounted in an instrument along with a GEx slide and a permeabilization buffer comprising sarkosyl and proteinase K. Upon sandwich closure in the instrument, the tissue sections were permeabilized for 1 minute. For the tissue-mounted GEx slides processed according to the non-sandwich condition, sections were permeabilized for 5 minutes using the same permeabilization buffer without sandwiching. For both conditions, following permeabilization, captured polyA-containing mRNA transcripts on the GEx slides were reverse transcribed into cDNA, followed by standard sequencing library preparation and sequencing.

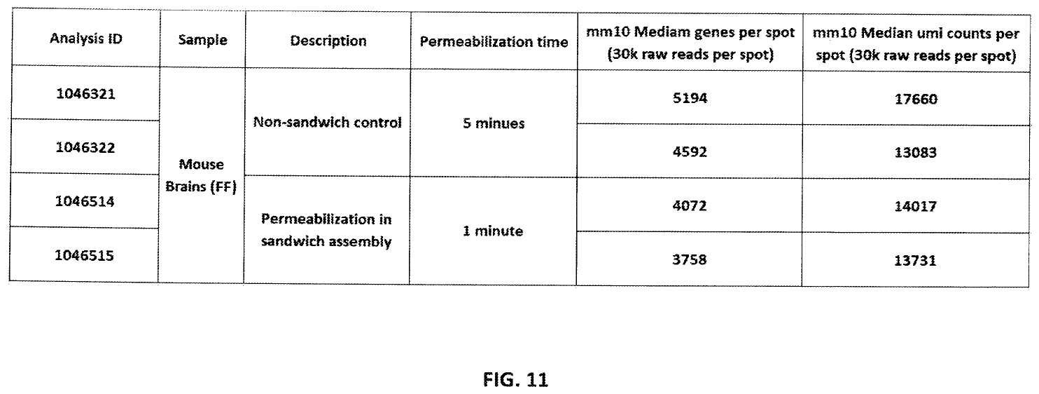

Results depicting median genes per spot and median UMI counts per spot are shown in FIG. 11.

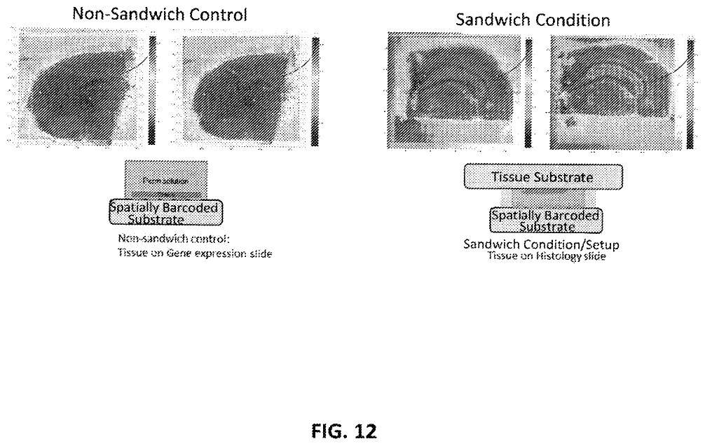

Visual heat map results showing Log 10 UMIs are shown in FIG. 12. Spatial patterns of the Log 10 UMI counts were similar across the sandwich and non-sandwich conditions.

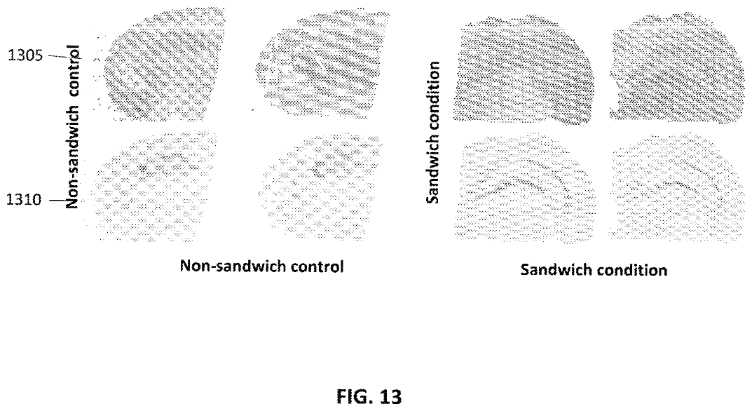

Spatial clustering analysis (top row 1305) and analysis of hippocampal transcript Hpca (bottom row 1310) are depicted in FIG. 13. Spatial patterns were comparable across the sandwich and non-sandwich conditions.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. A method for determining a location of multiple analytes in a biological sample, the method comprising:
(a) providing a first substrate and the biological sample mounted thereon, wherein the first substrate comprises a plurality of first capture probes, wherein a first capture probe of the plurality of first capture probes comprises (i) a first spatial barcode and (ii) a first capture domain;
(b) aligning a second substrate on the opposite side of the first substrate relative to the biological sample, thereby sandwiching the biological sample between the first and the second substrate, wherein the second substrate comprises a plurality of second capture probes, wherein a second capture probe of the plurality of second capture probes comprises (i) a second spatial barcode and (ii) a second capture domain;
(c) adding a reagent medium comprising a permeabilization buffer to the biological sample, thereby promoting migration of a first analyte and a second analyte or a second intermediate agent from the biological sample to the first substrate and/or the second substrate;
(d) hybridizing the first analyte to the first capture domain, and hybridizing the second analyte or the second intermediate agent to the second capture domain; and
(e)
(A) determining (i) the sequence of the first spatial barcode, or a complement thereof, and (ii) all or a portion of the sequence of the first analyte, or a complement thereof, and using the sequences of (i) and (ii) to determine the location of the first analyte in the biological sample, and
(B) determining (iii) the sequence of the second spatial barcode, or a complement thereof, and (iv) all or a portion of the sequence of the second analyte or the second intermediate agent, or a complement thereof, and using the sequences of (iii) and (iv) to determine the location of the second analyte in the biological sample.

2. The method of claim 1, further comprising, prior to (a), mounting the biological sample onto the plurality of first capture probes of the first substrate.

3. The method of claim 1, wherein the permeabilization buffer comprises pepsin or proteinase K.

4. The method of claim 1, wherein step (b) is performed using a sample holder comprising: (i) a first member comprising a first retaining mechanism configured to receive the first substrate, (ii) a second member comprising a second retaining mechanism configured to receive the second substrate, and (iii) an alignment mechanism that is connected to at least one of the first member and second member and configured to align the first substrate and the second substrate.

5. The method of claim 4, wherein step (b) comprises (i) retaining the first substrate in the first retaining mechanism of the first member, (ii) retaining the second substrate in the second retaining mechanism of the second member, and (iii) using the alignment mechanism to align the second substrate on the opposite side of the first substrate relative to the biological sample, thereby sandwiching the biological sample between the first substrate and the second substrate.

6. The method of claim 1, wherein the first capture domain comprises a sequence complementary to a portion of the first analyte.

7. The method of claim 1, wherein the plurality of first capture probes and/or the plurality of second capture probes are arranged on a plurality of beads, and/or wherein the first capture probe and/or the second capture probe further comprise a sequence selected from the group consisting of a functional domain, a unique molecular identifier, a cleavage domain, and a combination thereof.

8. The method of claim 1, further comprising generating an extended first capture probe using the first analyte as a template, and/or generating an extended second capture probe using the second analyte or second intermediate agent as a template.

9. The method of claim 8, further comprising amplifying the extended first capture probe and/or the extended second capture probe to produce a plurality of extended capture probes.

10. The method of claim 1, wherein the determining step in step (e) (A) comprises sequencing (i) the first spatial barcode or the complement thereof, and (ii) all or a portion of the sequence of the first analyte, or the complement thereof, and/or the determining step in step (e) (B) comprises sequencing (i) the second spatial barcode or the complement thereof, and (ii) all or a portion of the sequence of the second analyte or the second intermediate agent, or the complement thereof.

11. The method of claim 1, wherein the second intermediate agent comprises a second capture handle sequence, wherein the method further comprises contacting the biological sample with a plurality of analyte capture agents comprising a second analyte capture agent, wherein:
the second analyte capture agent comprises a second analyte binding moiety and a second capture agent barcode domain, wherein the second analyte binding moiety binds to the second analyte, and wherein the second capture agent barcode domain comprises a second analyte binding moiety barcode and the second capture handle sequence; wherein the second intermediate agent is generated by removing the second capture agent barcode domain from the second analyte capture agent.

12. The method of claim 11, wherein the second analyte is a protein analyte.

13. The method of claim 11, wherein the second analyte binding moiety comprises an antibody or an antigen-binding fragment thereof.

14. The method of claim 11, wherein the second analyte capture agent comprises a linker that couples the second capture agent barcode domain to the second analyte binding moiety.

15. The method of claim 14, wherein the linker is a cleavable linker comprising a disulfide linker, a photo-cleavable linker, a UV-cleavable linker, or an enzyme cleavable linker.

16. The method of claim 1, wherein the second intermediate agent comprises a second connected probe, and wherein the method further comprises:
hybridizing a third probe and a fourth probe to the second analyte, wherein the third probe and the fourth probe each comprise a sequence that is substantially complementary to adjacent sequences of the second analyte, and wherein the fourth probe comprises a second capture probe binding domain; and
coupling the third probe and the fourth probe, thereby generating a second connected probe.

17. The method of claim 1, wherein the first analyte and the second analyte are RNA molecules.

18. The method of claim 1, wherein the first analyte is a nucleic acid and the second analyte is a protein.

19. The method of claim 1, wherein the biological sample is a formalin-fixed, paraffin-embedded tissue section, a frozen tissue section, a fresh frozen tissue section, or a fresh tissue section.

20. The method of claim 1, further comprising staining, imaging, or destaining the biological sample, or a combination thereof.
